(19) United States
(12) Patent Application Publication
(10) Pub. No.: US 2009/0176743 A1
(43) Pub. Date: Jul. 9, 2009

(54) Methods for treating or preventing reactivation of a latent herpesvirus infection

(76) Inventors: Priscilla Schaffer, Holliston, MA (US); Ryan Bringhurst, Westborough, MA (US)

Correspondence Address: CLARK & ELBING LLP, 101 FEDERAL STREET, BOSTON, MA, 02110, US

(21) Appl. No.: 11/989,700

(22) PCT Filed: Jul. 31, 2006
(86) PCT No.: PCT/US06/29663

§ 371 Date: Feb. 25, 2008

Related U.S. Application Data
(60) Provisional application No. 60/703,835, filed on Jul. 29, 2005.

(51) Int. Cl. A61K 31/195; A61K 31/675; A61P 31/12

(52) U.S. Cl. 514/81; 514/561; 514/565; 514/562; 514/79

(57) ABSTRACT

The invention is directed to methods and compositions for treating or preventing reactivation of a latent herpesvirus infection and the associated complications and outcomes. The methods involve administering a composition comprising glutamine, or a derivative, conjugate, or analog thereof.

### BACKGROUND OF THE INVENTION

The invention relates to the treatment or prevention of herpes family viral reactivation in infected patients.

Herpesviruses are DNA viruses and among them are herpes simplex virus type 1 (HSV-1), herpes simplex virus type 2 (HSV-2), varicella-zoster virus (VZV), cytomegalovirus (CMV), Epstein-Barr virus (EBV), and various other human herpesviruses (HHV), such as HHV-6, HHV-7, and HHV-8. Herpesviruses are, in general, transmitted by person-to-person contact by infected body secretions, and infection by a herpesvirus can cause various diseases in humans. The severity of infection depends on the virus type and the immune status of the infected host. Primary and recurrent infections can sometimes be relatively mild, but under certain circumstances can be fatal to the host.

Primary HSV-1 and HSV-2 infections occur through a break in the mucus membranes of the mouth or throat, via the eye or genitals, or directly via minor abrasions in the skin. Because of the universal distribution of the virus, most individuals are infected by some type of herpesvirus by 1 to 2 years of age; initial infection is sometimes asymptomatic, although there may be minor local vesicular lesions. Local multiplication ensues. There then follows life-long latent infection, particularly of noncycling neurons, with periodic reactivation. The latency period is characterized by minimal viral gene expression, the absence of new virus synthesis, and neuron viability. Periodically, HSV-1 and HSV-2 reactivate to cause recurrent disease. Recurrence is common, and may be stimulated by various physiological stresses, trauma, emotional stress, and hormonal changes. Particularly stressful stimuli are fever and sunburn, which are known to promote reactivation of latent herpesviruses and synthesis of low levels of new infectious virus.

HSV-1 and HSV-2 produce a variety of infections involving vesicular eruptions on the skin and mucus membranes, and may also affect the central nervous system and occasionally visceral organs. HSV-1 infection is associated mainly with the oral region (oral herpes) and causes cold sores and fever blisters. HSV-2 causes lesions that are similar to oral herpes, but that occur mainly in the genital region (genital herpes). Once herpesviruses have infected the host, they persist in these host cells. For example, after infecting epithelial cells, herpes simplex viruses secondarily invade nerve tissues and remain latent in these cells for the lifetime of the host. With HSV-1, latency occurs in facial nerve tissue (e.g., the trigeminal ganglion). HSV-2 establishes latency in the sacral ganglia, which are in the pelvic region.

In addition to its primary local pathogenesis at the site of infection, HSV-1 is associated with the incidence of other health problems, such as viral encephalitis. HSV-2 is a particularly important public health problem with the overall prevalence of HSV-2 virus in the population estimated to be between 10% and 70%, and even as high as 80% in some populations. Additionally, if HSV-2 infection is transmitted to newborns during birth, the subsequent infection may be devastating. A significant percentage of newborns delivered by women with genital herpes become infected with HSV-2; many of these infants suffer severe virus-induced defects, which can include retardation. There are numerous associations between herpesvirus infections and the contracting or development of other serious diseases. For example, HSV-2 infection has been found to be a risk factor for the acquisition and transmission of infection of human immunodeficiency virus type 1 (HIV-1), which is a causative factor of acquired immunodeficiency syndrome (AIDS). HSV infections are particularly severe and even life-threatening to patients with AIDS. Only 20 percent of herpes seropositive persons have symptomatic infection; the rest are asymptomatic but shed the virus, which can result in new infections in those individuals that come into contact with the infectious individual.

Other members of the herpesvirus family that have been implicated in disease in humans include, for example, CMV. CMV has been shown to cause eye infections, which can result in blindness if left untreated. Infection of immuno-compromised patients with CMV can result in significant morbidity and mortality. Another herpesvirus, VZV, is the causative agent of chicken pox upon primary infection and zoster when it recurs in adults. Zoster is associated with dermatomal vesicular rash or shingles that can be quite painful. EBV results in approximately two million cases of infectious mononucleosis in the United States each year.

In the past, treatment for HSV-1 and HSV-2 lesions consisted primarily of topical application of drugs for symptomatic relief, such as analgesics and anesthetics for the relief of pain, which had minimal therapeutic effect on the lesions. Also, various treatments involving painting of the lesions with acridine dyes and then exposing them to ultraviolet light have been tried without significant therapeutic effect, and with an associated risk of producing malignant cells. More recent methods have been proposed and used as treatments for herpes infection, including the administration of various pharmaceuticals, such as iododeoxyuridine, adenine arabinoside (ara-A) and acycloguanosine (acyclovir). Iododeoxyuridine and ara-A are used to treat HSV-1 eye infections, while ara-A may reduce the severity of encephalitis caused by HSV-1 and HSV-2 infection of newborns. Acyclovir is currently considered to be the mainstay of drug therapy in the treatment of herpes, both genital and oral. However, none of these methods has proved to be entirely effective. For example, while acyclovir has been shown to speed the healing and resolution of genital herpes infections, the benefit of treating acute episodes of recurrent genital disease is quite modest and not recommended as a long-term therapy. Acyclovir has a very limited benefit with regards to oral herpes; in many cases, developing lesions are not aborted and healing time is not reduced. Most seriously, acyclovir-resistant strains of herpesviruses are being identified with increasing frequency, especially in individuals infected with HIV. Thus, there is a need to develop new compositions and methods for treating herpesvirus infections that are effective, safe, and practical.

Glutamine is the most abundant of the amino acids in the human body. Its main storage site is in the musculature, where about 60% of all the unbound amino acids are glutamine (glutamine makes up a smaller percentage of muscle protein, the main bound form). Glutamine is manufactured in the body from glutamate and ammonia by the enzyme glutamine synthetase; the process takes place mainly in the skeletal muscles. The amount of glutamine in reserve for release as needed is directly related to muscle mass: more muscle mass means more glutamine is available for metabolic processes. Under conditions of metabolic stress, including injuries, illness, and even severe emotional distress, the level of glutamine in the body declines markedly, which is thought to adversely influence resistance to infectious diseases. Persons who maintain a relatively large muscle mass may have a greater ability to withstand and recover from stressful events. Chronic illness and lack of exercise work together in a vicious cycle: poor health makes it more difficult to exercise, leading to lower muscle mass and lower glutamine stores, contributing to a higher incidence of health problems and slower recovery.

There exists a need for therapeutics to treat and prevent the recurrence of herpesvirus lesions for reasons of individual and public health. Such therapeutics would aid the affected individual and decrease transmission to previously uninfected individuals.

### SUMMARY OF THE INVENTION

The present invention relates to methods and compositions for the treatment or prevention of herpesvirus reactivation and the diseases or conditions caused by reactivation of latent herpesvirus infection. Clinically, some of the goals of treatment or prevention include reducing the severity of disease associated with primary infection; reducing the frequency of reactivation of latent virus; limiting the severity of reactivated disease; and restricting the transmission of virus associated with either primary or reactivated infection(s). The compositions of the invention include glutamine, or conjugates or analogs thereof, in a pharmaceutically acceptable carrier. The compositions can be administered for treating or preventing reactivation of herpes viral infections or the diseases or conditions caused therewith, including conditions caused by HSV-1, such as cold sores; HSV-2, such as genital herpes; as well as shingles caused by VSV and infections caused by CMV and EBV.

A first aspect of the invention features a method for treating or preventing the reactivation of a latent herpesvirus infection in a subject (e.g., a human) by administering to the subject a therapeutically effective amount of a composition containing glutamine, or a derivative, conjugate, or analog thereof. In several embodiments of the first aspect, the latent herpesvirus infection is an infection caused by a herpesvirus selected from a group consisting of herpes simplex virus (HSV) type 1 (HSV-1), HSV-2, cytomegalovirus (CMV), Epstein Barr virus (EBV), varicella zoster virus (VZV), human herpes virus (HHV)-6, HHV-7, and HHV-8. In another embodiment of the first aspect of the invention, the latent herpesvirus infection is an infection of the skin, a mucous membrane, or the neurological system. In another related embodiment, the infection is an infection of the eyes, mouth, lips, genital area, or anal area of the subject.

In a related embodiment of the first aspect of the invention, the administering is performed by injection, oral administration, or topical administration.

The composition can also include arginine, methionine, or arginine and methionine, or derivatives, conjugates, or analogs thereof. The method can include administering to the subject a separate composition that includes arginine, methionine, or arginine and methionine, or derivatives, conjugates, or analogs thereof. In another related embodiment, the method further includes administering a composition that includes an inhibitor of herpesvirus thymidine kinase (e.g., azidodeoxythymidine, didehydrodeoxythymidine, 2-phenylamino-9-substituted-6-oxopurines, and 2-phenylamino-9H-6-oxopurines, or a ester, salt, or solvate thereof), lysine, or an antiherpesvirus substance selected from a pre-phosphorylated or phosphonate nucleoside analog (e.g., acyclovir monophosphate, ganciclovir monophosphate, and 9-(phosphonomethoxyethyl)adenine (PMEA), or an ester, salt, or solvate thereof), a pyrophosphate analog (e.g., phosphonoacetate and phosphonoformate), and a nucleoside analog (e.g., acyclovir, ganciclovir, cidofovir, and famcyclovir), or any combination thereof, or an ester, salt, or solvate thereof. In another related embodiment, the composition containing glutamine further includes an inhibitor of herpesvirus thymidine kinase (e.g., azidodeoxythymidine, didehydrodeoxythymidine, 2-phenylamino-9-substituted-6-oxopurines, and 2-phenylamino-9H-6-oxopurines, or a ester, salt, or solvate thereof), lysine, or an antiherpesvirus substance selected from a pre-phosphorylated or phosphonate nucleoside analog (e.g., acyclovir monophosphate, ganciclovir monophosphate, and 9-(phosphonomethoxyethyl)adenine (PMEA), or an ester, salt, or solvate thereof), a pyrophosphate analog (e.g., phosphonoacetate and phosphonoformate), and a nucleoside analog (e.g., acyclovir, ganciclovir, cidofovir, and famcyclovir), or any combination thereof, or an ester, salt, or solvate thereof. In another embodiment, the composition includes a pharmaceutically acceptable carrier. In yet another embodiment, the composition is administered in a therapeutically effective amount topically to an area of the body (e.g., the eyes, mouth, lips, genital area, anal area, and mixtures thereof). In another embodiment of the first aspect of the invention, the method includes diagnosing the subject with a herpesvirus infection prior to administering.

A second aspect of the invention features a composition having an amount of glutamine that is sufficient for preventing or reducing reactivation of a latent herpesvirus infection in combination with a pharmaceutically acceptable carrier. In an embodiment of the second aspect of the invention, the composition is packaged for parenteral, oral, or topical use by a patient. In another embodiment, the packaging further includes instructions for the administration of the composition for treating or preventing reactivation of a herpesvirus infection. In other, related embodiments, the composition can be formulated as a cream, lotion, gel, ointment, plaster, stick, pen, injection, or tablet. In other embodiments, the pharmaceutically acceptable carrier is selected from sterile water, saline, polyalkylene glycols, vegetable oils, hydrogenated naphthalenes, biocompatible polymers, biodegradable polymers (e.g., polycaprolactone, polydecalactone, poly(sebacic anhydride), sebacic acid-co-1,3-bis(carboxyphenoxypropane), sebacic acid-co-1,6-bis(carboxyphenoxyhexane), dedecanoic-co-1,3-bis(carb-oxyphenoxypropane), dedecanoic-co-1,6-bis(carboxyphenoxyhexane), albumin and derivatives, gelatin and derivatives, starch and derivatives, gum arabic, cellulose and derivatives, dextran and derivatives, polysorbate and derivatives, agarose, lectins, galactose, polyurethanes, polyvinylalcohol, functionalized polymers and copolymers of lactic and glycolic acid, lactic acid homopolymer, glycolic acid copolymer, copolymers of lactic acid and glycolic acid, polyhydroxybutyrate, polyhydroxyalkanoic acid, and mixtures thereof), and mixtures thereof.

In a related embodiment, the composition further includes arginine, methionine, arginine and methionine, or derivatives, conjugates, or analogs thereof. In other, related embodiments of the second aspect of the invention, the composition further includes an inhibitor of herpesvirus thymidine kinase (e.g., azidodeoxythymidine, didehydrodeoxythymidine, 2-phenylamino-9-substituted-6-oxopurines, and 2-phenylamino-9H-6-oxopurines, or a ester, salt, or solvate thereof), lysine, or an antiherpesvirus substance selected from a pre-phosphorylated or phosphonate nucleoside analog (e.g., acyclovir monophosphate, ganciclovir monophosphate, and 9-(phosphonomethoxyethyl)adenine (PMEA), or an ester, salt, or solvate thereof), a pyrophosphate analog (e.g., phosphonoacetate and phosphonoformate), and a nucleoside analog (e.g., acyclovir, ganciclovir, cidofovir, and famcyclovir), or any combination thereof, or an ester, salt, or solvate thereof.

A third aspect of the invention features a method for treating or preventing the reactivation of a latent herpesvirus infection in a subject (e.g., a human) by administering to the subject a therapeutically effective amount of a composition containing arginine, methionine, or both, or derivatives, conjugates, or analogs thereof. In several embodiments of the third aspect, the latent herpesvirus infection is an infection caused by a herpesvirus selected from a group consisting of herpes simplex virus (HSV) type 1 (HSV-1), HSV-2, cytomegalovirus (CMV), Epstein Barr virus (EBV), varicella zoster virus (VZV), human herpes virus (HHV)-6, HHV-7, and HHV-8. In another embodiment of the third aspect of the invention, the latent herpesvirus infection is an infection of the skin, a mucous membrane, or the neurological system. In another related embodiment, the infection is an infection of the eyes, mouth, lips, genital area, or anal area of the subject.

In a related embodiment of this aspect of the invention, the administering is performed by injection, oral administration, or topical administration.

The composition can also include glutamine, or derivatives, conjugates, or analogs thereof. The method can include administering to the subject a separate composition that includes glutamine; arginine and methionine; glutamine and arginine; or glutamine and methionine, or derivatives, conjugates, or analogs thereof. In another related embodiment, the method further includes administering a composition that includes an inhibitor of herpesvirus thymidine kinase (e.g., azidodeoxythymidine, didehydrodeoxythymidine, 2-phenylamino-9-substituted-6-oxopurines, and 2-phenylamino-9H-6-oxopurines, or a ester, salt, or solvate thereof), lysine, or an antiherpesvirus substance selected from a pre-phosphorylated or phosphonate nucleoside analog (e.g., acyclovir monophosphate, ganciclovir monophosphate, and 9-(phosphonomethoxyethyl)adenine (PMEA), or an ester, salt, or solvate thereof), a pyrophosphate analog (e.g., phosphonoacetate and phosphonoformate), and a nucleoside analog (e.g., acyclovir, ganciclovir, cidofovir, and famcyclovir), or any combination thereof, or an ester, salt, or solvate thereof. In another related embodiment, the composition containing arginine or methionione or arginine and methionine further includes an inhibitor of herpesvirus thymidine kinase (e.g., azidodeoxythymidine, didehydrodeoxythymidine, 2-phenylamino-9-substituted-6-oxopurines, and 2-phenylamino-9H-6-oxopurines, or a ester, salt, or solvate thereof), lysine, or an antiherpesvirus substance selected from a pre-phosphorylated or phosphonate nucleoside analog (e.g., acyclovir monophosphate, ganciclovir monophosphate, and 9-(phosphonomethoxyethyl)adenine (PMEA), or an ester, salt, or solvate thereof), a pyrophosphate analog (e.g., phosphonoacetate and phosphonoformate), and a nucleoside analog (e.g., acyclovir, ganciclovir, cidofovir, and famcyclovir), or any combination thereof, or an ester, salt, or solvate thereof. In another embodiment, the composition includes a pharmaceutically acceptable carrier. In yet another embodiment, the composition is administered in a therapeutically effective amount topically to an area of the body (e.g., the eyes, mouth, lips, genital area, anal area, and mixtures thereof). In another embodiment of the first aspect of the invention, the method includes diagnosing the subject with a herpesvirus infection prior to administering.

A fourth aspect of the invention features a composition having an amount of arginine or methionine or both that is sufficient for preventing or reducing reactivation of a latent herpesvirus infection in combination with a pharmaceutically acceptable carrier. In an embodiment of the second aspect of the invention, the composition is packaged for parenteral, oral, or topical use by a patient. In another embodiment, the packaging further includes instructions for the administration of the composition for treating or preventing reactivation of a herpesvirus infection. In other, related embodiments, the composition can be formulated as a cream, lotion, gel, ointment, plaster, stick, pen, injection, or tablet. In other embodiments, the pharmaceutically acceptable carrier is selected from sterile water, saline, polyalkylene glycols, vegetable oils, hydrogenated naphthalenes, biocompatible polymers, biodegradable polymers (e.g., polycaprolactone, polydecalactone, poly(sebacic anhydride), sebacic acid-co-1,3-bis(carboxyphenoxypropane), sebacic acid-co-1,6-bis(carboxyphenoxyhexane), dedecanoic-co-1,3-bis(carb-oxyphenoxypropane), dedecanoic-co-1,6-bis(carboxyphenoxyhexane), albumin and derivatives, gelatin and derivatives, starch and derivatives, gum arabic, cellulose and derivatives, dextran and derivatives, polysorbate and derivatives, agarose, lectins, galactose, polyurethanes, polyvinylalcohol, functionalized polymers and copolymers of lactic and glycolic acid, lactic acid homopolymer, glycolic acid copolymer, copolymers of lactic acid and glycolic acid, polyhydroxybutyrate, polyhydroxyalkanoic acid, and mixtures thereof), and mixtures thereof.

In a related embodiment, the composition further includes glutamine, or derivatives, conjugates, or analogs thereof. In other, related embodiments of this aspect of the invention, the composition further includes an inhibitor of herpesvirus thymidine kinase (e.g., azidodeoxythymidine, didehydrodeoxythymidine, 2-phenylamino-9-substituted-6-oxopurines, and 2-phenylamino-9H-6-oxopurines, or a ester, salt, or solvate thereof), lysine, or an antiherpesvirus substance selected from a pre-phosphorylated or phosphonate nucleoside analog (e.g., acyclovir monophosphate, ganciclovir monophosphate, and 9-(phosphonomethoxyethyl)adenine (PMEA), or an ester, salt, or solvate thereof), a pyrophosphate analog (e.g., phosphonoacetate and phosphonoformate), and a nucleoside analog (e.g., acyclovir, ganciclovir, cidofovir, and famcyclovir), or any combination thereof, or an ester, salt, or solvate thereof.

In an embodiment of any of the aspects of the invention, methods are provided for using the compositions of the invention for the treatment of herpesvirus infections in human patients. For example, HSV1, HSV2, VZV, and EBV viral infections causing diseases such as oral herpes, genital herpes, encephalitis, and shingles may be treated.

In various embodiments of the invention, the subject to be treated is an animal, e.g., a mammal. In some cases the mammal may be a human, horse, cow, pig, dog, or mouse. Alternatively, the subject of the methods of the invention may be selected from chickens, turtles, lizards, fish and other animals susceptible to herpesvirus infection. In preferred embodiments, the animal or subject is a human.

The compounds of the invention are particularly useful for preventing viral reactivation in individuals infected with herpesviruses, such as HSV1, HSV2, HHV6, HHV7, HHV8, VZV, CMV, and EBV.

By “administration” or “administering” is meant a method of giving a dosage of a pharmaceutical composition containing glutamine, or a conjugate, derivative, or analog thereof, to a mammal, where the method is, e.g., topical, oral, intravenous, intraperitoneal, intracranial, intravenous, intra-arterial, or intramuscular. The preferred method of administration can vary depending on various factors, e.g., the components of the pharmaceutical composition, site of the potential or actual disease, and severity of disease.

By “analog” is meant a molecule that differs from, but is structurally, functionally, and/or chemically related to the reference molecule (i.e., glutamine). The analog may retain the essential properties, functions, or structures of the reference molecule. Most preferably, the analog retains at least one biological function of the reference molecule. Generally, differences are limited so that the structure or sequence of the reference molecule and the analog are similar overall. An analog of glutamine may be naturally occurring, or it may be a variant that is not known to occur naturally. Non-naturally occurring analogs of glutamine may be made synthetically or by modification.

By “carrier” is meant any and all appropriate solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

By “conjugate” is meant a compound formed as a composite between two or more molecules. More specifically, in the present invention, a glutamine conjugate is a compound in which glutamine is bonded, for example, covalently bonded, to an independent moiety forming a conjugate compound. The moiety can include a therapeutic or diagnostic agent, e.g., a targeting compound, a cytotoxic agent, a detectable labels, or a chelating group.

By “cytotoxic agent” is meant any naturally occurring, modified, or synthetic compound that is toxic to cells. Cytotoxic agents include, but are not limited to, alkylating agents, antibiotics, antimetabolites, tubulin inhibitors, topoisomerase I and II inhibitors, hormonal agonists or antagonists, or immunomodulators. They may also be cytotoxic when activated by light or infrared (Photofrin, IR dyes; Nat. Biotechnol. 19(4):327-331, 2001). They may operate through other mechanistic pathways, or cytoxic agents may also be supplementary potentiating agents.

By “detectable label” is meant any type of label which, when attached to a peptide agent, renders the compound detectable. A detectable label may be toxic or non-toxic, and may have one or more of the following attributes, without restriction: fluorescence (Kiefer et al., WO 9740055), color, toxicity (e.g., radioactivity, e.g., a γ-emitting radionuclide, Auger-emitting radionuclide, β-emitting radionuclide, an α-emitting radionuclide, or a positron-emitting radionuclide), radiosensitivity, or photosensitivity. Although a detectable label may be directly attached to an amino acid residue, a detectable label may also be indirectly attached, for example, by being complexed with a chelating group that is attached (e.g., linked via a covalent bond or indirectly linked) to an amino acid residue. A detectable label may also be indirectly attached to an analog by the ability of the label to be specifically bound by a second molecule. One example of this type of an indirectly attached label is a biotin label that can be specifically bound by the second molecule, streptavidin. The second molecule may also be linked to a moiety that allows neutron capture (e.g., a boron cage as described in, for example, Kahl et al., Proc. Natl. Acad. Sci. USA 87:7265-7269, 1990).

A detectable label may also be a metal ion from heavy elements or rare earth ions, such as Gd³⁺, Fe³⁺, Mn³⁺, or Cr²⁺ (see, for example, Invest. Radiol. 33(10):752-761, 1998). Preferred radioactive detectable labels are radioactive iodine labels (e.g., ¹²²I, ¹²³I, ¹²⁴I, ¹²⁵I, or ¹³¹I) that are capable of being coupled to a glutamine residue of the invention. Preferred non-radioactive detectable labels are the many known dyes that are capable of being coupled to NH₂-terminal amino acid residues.

Preferred examples of detectable labels include ricin, diptheria toxin, and radioactive detectable labels (e.g., ¹²²I, ¹²³I, ¹²⁴I, ¹²⁵I, ¹³¹I, ¹⁷⁷Lu, 64Cu, ⁶⁷Cu, ¹⁵³Sm, ¹⁶⁶Ho, ¹⁸⁶Re, ¹⁸⁸Re, ²¹¹At, ²¹²Bi, ²²⁵Ac, ⁶⁷Ga, ⁶⁸Ga, ⁷⁵Br, ⁷⁶Br, ⁷⁷Br, ^{117m}Sn, ⁴⁷Sc, ¹⁰⁹Pd, ⁸⁹Sr, ¹⁵⁹Gd, ¹⁴⁹Pm, ¹⁴²Pr, ¹¹¹Ag, ¹⁶⁵Dy, ²¹³Bi, ¹¹¹In, ^{114m}In, ²⁰¹Ti, ^{195m}Pt, ¹⁹³Pt, ⁸⁶Y and ⁹⁰Y. These compounds, and others described herein may be directly or indirectly attached to glutamine, or a derivative or analog thereof. A toxic detectable label may also be a chemotherapeutic agent (e.g., camptothecins, homocamptothecins, 5-fluorouracil or adriamycin), or may be a radiosensitizing agent (e.g., Taxol, gemcitabine, fluoropyrimidine, metronitozil, or the deoxycytidine analog 2′,2′ difluoro-2′-deoxycytidine (dFdCyd).

By “chelating group” is meant any group covalently bound to glutamine, or a derivative or analog thereof, that may complex with a detectable label, such as a metal, photosensitizing agent, etc. Chelating groups, for example, include an iminodicarboxylic group or a polyaminopolycarboxylic group. Chelating groups may be attached using the methods generally described in Liu et al., Bioconjugate Chem. 12(4):653, 2001; Alter et al., U.S. Pat. No. 5,753,627; and PCT Publication No. WO 91/01144; both of which are hereby incorporated by reference). A glutamine analog of the invention may be complexed, through its attached chelating agent, to a detectable label, thereby resulting in an analog that is indirectly labeled. Similarly, cytotoxic or therapeutic agents, may also be attached via a chelating group to a peptide agent of the invention.

By “pharmaceutically-acceptable” is meant any molecular entity or composition that does not produce an allergic or similar untoward reaction when administered to a human.

By “therapeutically effective amount” is meant an amount of a composition containing glutamine, or a derivative, conjugate, or analog thereof, sufficient to reduce or prevent the reactivation of, or to decrease the frequency of reactivation of, a herpesvirus. The term “therapeutically effective amount” therefore includes, for example, an amount of a composition containing glutamine, or a derivative, conjugate, or analog thereof (either with or without one or more additional anti-herpes compounds) that is sufficient to prevent the reactivation of herpesvirus or to reduce the reactivation of a herpesvirus in an infected cell. In preferred embodiments, the reduction in reactivation of herpesvirus is by at least 50%, and more preferably at least 60%, 70%, 80%, or 90%, and most preferably by at least 95% or more. The dosage ranges for the administration of the glutamine composition are those that produce the desired effect. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the reactivation of the herpesvirus by methods well known to those in the field, and as described below.

By “treating” is meant curing or ameliorating a disease or condition caused by reactivation of a herpesvirus infection or tempering the severity of a disease or condition associated with reactivation of a herpesvirus infection. By “preventing” is meant blocking or reducing the reactivation of a latent virus of the herpes family and blocking or reducing a disease or condition caused by such reactivation (e.g., the formation of a primary lesion or blister caused by recurrence at a herpesvirus infection site). The dosages of the glutamine composition, which can treat or prevent reactivation of a herpesvirus infection can be determined in view of this disclosure by one of ordinary skill in the art by running routine trials with appropriate controls. Comparison of the appropriate treatment groups to the controls will indicate whether a particular dosage is effective in preventing or treating the infection at the levels used in a controlled challenge.

As used herein, “treatment” includes, but is not limited to, ameliorating a disease, lessening the severity of its complications, preventing it from manifesting, preventing it from recurring, merely preventing it from worsening, mitigating an inflammatory response included therein, or a therapeutic effort to affect any of the aforementioned, even if such therapeutic effort is ultimately unsuccessful.

Other features and advantages of the invention will be apparent from the following Detailed Description, the drawings, and the claims.

### BRIEF DESCRIPTION OF THE DRAWINGS

FIGS. 1A-1C are graphs demonstrating that cellular heat shock prior to infection results in enhanced plating efficiency of an ICP0-herpesvirus versus a wild type (KOS) herpesvirus strain. FIG. 1A shows the effect of heat shock at 37° C. FIG. 1B shows the effect of heat shock at 41° C. FIG. 1C shows the effect of heat shock at 43° C.

FIGS. 2A-2F are a series of graphs showing that cellular heat shock enhances the replication efficiency of an ICP0⁻ virus in an MOI-dependent manner. The top row (FIGS. 2A, C, and E) represents viral yields, and the bottom row (FIGS. 2B, D, and F) represents fold change in replication efficiency following heat shock, calculated by dividing PFU/cell following heat shock by PFU/cell without heat shock. The figure is representative of two experiments, which produced similar results.

FIGS. 3A-C are a series of graphs showing that the heat shock-induced induced increase in plating efficiency of ICP0⁻ viruses on Vero cell monolayers is transient. Times shown on the x-axis are the number of hours following transfer to the elevated temperature. This experiment was performed twice for KOS and 7134 and once for n212. For KOS and 7134, the figure represents the average of the two experiments, and error bars represent the standard deviation.

FIGS. 4A-4B are graphs showing that UV-C irradiation of Vero cells prior to infection results in enhanced plating efficiency of an ICP0-herpesvirus versus wild type (KOS) herpesvirus. FIG. 4A shows the PFU produced per well over time. FIG. 4B shows the fold change in plating efficiency with respect to time when the Vero cells are exposed to increasing levels of UV-C irradiation.

FIGS. 5A-5C are graphs showing the plating efficiencies of an ICP0 null virus and a wild type (KOS) virus on Vero cell monolayers synchronized by isoleucine deprivation. Twenty-four-hour-old Vero cell monolayers were harvested and seeded in 60-mm-diameter Petri dishes (5×10⁵ cells per dish). Monolayers were maintained in isoleucine free medium from 24 to 66 h postseeding. Cells were then released from the block by growth in medium containing isoleucine. FIG. 5A is a graph showing the fraction of Vero cells at various stages of mitosis post-release. FIG. 5B is a graph showing the number of cells per dish. FIG. 5C is a graph showing the virus titer following infection of the Vero cells with 40 PFU of ICP0 null virus or 100 PFU of the wild type virus (input multiplicities of infection were based on titers determined on 25-h-old Vero cells). FIG. 5C shows that the plating efficiency of ICP0 null virus is greatest on Vero monolayers 8 h after release from growth arrest.

FIGS. 6A-6D are a series of graphs showing that the plating efficiency of ICP0⁻ viruses decreases with increasing cell density rather than increasing age of the monolayers, and heat shock greatly enhances plating efficiency on very dense monolayers. The number of PFU/plate was plotted against the number of cells/plate at the time of infection. The number of hours post-plating, though not used to generate the plots, is also shown and corresponds to the time of infection. The average number of plaques/plate corresponds to the cell number at the indicated times; error bars indicate the standard deviation of 3 independent experiments.

FIGS. 7A-7H are graphs showing that the plating efficiency of an ICP0 null virus is enhanced only in media that lacks glutamine, but that the plating efficiency is not enhanced at certain stages of the cell cycle. FIGS. 7A-7D are graphs showing the fraction of Vero cells in the G₁, S, and G₂M phase of the cell cycle following incubation in media lacking isoleucine (Ile−) or fetal bovine serum (FBS−), and also containing or lacking glutamine (Gln+ vs. Gln−). FIGS. 7E-7H are graphs showing the number of PFU produced per plate during a 24 h incubation in the indicated media.

FIG. 8 is a graph showing that the plating efficiency of the ICP0 null virus is greatly enhanced when Ile-free medium also lacks Gln. The plating efficiency is slightly enhanced when the Gln concentration is below 1 mM and increases as Gln concentrations decrease. Comparing this experiment to the results at the 8-h time point in FIG. 5C suggests that the high virus titer was produced at a time when the Gln present in the medium was less than 0.25 mM.

FIG. 9 is a graph showing that the plating efficiency of the ICP0 null virus is greatly enhanced when Vero cells are incubated 42 h in the absence of Gln. Lysine deprivation and refeeding caused a slight enhancement compared with the 2^{nd} lane in which cells were treated with isoleucine deprivation and refeeding.

FIG. 10 is a graph showing the enhanced plating efficiency of ICP0 null viruses when glutamine concentration is less than 0.5 mM. Glutamine concentration is shown on the X-axis.

FIGS. 11A-B are graphs showing that the enhanced ICP0 null virus plating efficiency is due to glutamine deprivation rather than Ile deprivation. FIG. 11A shows the PFU/well for wild-type virus and FIG. 11B shows the PFU/well for ICP0 null virus.

FIG. 12 is a graph showing that the presence of high concentrations of glutamine in the culture medium decreases the plating efficiency of a wild type herpesvirus under stress conditions. The concentrations of glutamine are indicated on the x-axis. For Vero cell monolayers incubated at 43° C. for 1 or 2 hours, the presence of 20 mM glutamine slightly decreased the plating efficiency of the virus.

FIGS. 13A-B are graphs showing that high concentrations of glutamine did not prevent the enhanced plating efficiency of the ICP0 null virus due to heat shock. Presence and duration of heat shock is indicated on the X-axis. FIG. 13A shows the PFU/well for wild-type virus and FIG. 13B shows the PFU/well for ICP0 null virus.

FIG. 14 is a series of graphs showing that glutamine deprivation and to a lesser extent arginine and methionine deprivation resulted in enhanced plating efficiency of the ICP0 null virus over time. Deprivation of the other 12 amino acids had little effect.

FIGS. 15A-B are graphs showing the synergistic effect of arginine and methionine deprivation that enhances the plating efficiency of the ICP0 null virus at least as much as glutamine deprivation. FIG. 15A shows the PFU/well for wild-type virus and FIG. 15B shows the PFU/well for ICP0 null virus.

FIG. 16 is a graph showing that glutamine deprivation induces reactivation of wild-type virus from latency in the mouse ocular/trigeminal ganglia (TG) cell culture model. The y-axis above indicates the percentage of wells for each treatment (n=24) positive for cytopathic effect. TG incubated in 0 mM Gln reactivated more rapidly and to the same extent as heat-shocked TG. TG incubated in the presence of 2 mM Gln exhibited significantly reduced reactivation.

### DETAILED DESCRIPTION

The present invention relates to methods and compositions for the treatment or prevention of reactivation by a latent virus of the herpes family and the diseases or conditions caused by such reactivation. The inventors discovered that high concentrations of glutamine, or a derivative, conjugate, or analog thereof, can prevent or reduce the reactivation of a latent herpesvirus, particularly during stress conditions. Thus, the administration of a glutamine-containing composition to a patient can treat or prevent diseases or conditions associated with herpesvirus reactivation.

Difference Between Productive Infection of a Herpesvirus and Latency

Cellular factors induced by stress appear to play a decisive role in causing reactivation of herpesvirus from latency. Several important distinctions exist between productive infection and reactivation from latency. For example, during HSV-1 productive infection of cycling cells, VP16 and ICP0, two virus-specified proteins present in virions, both of which are potent transactivators of viral and cellular genes, are present and active. Therefore, during productive infection the virus actively controls the expression of viral and cellular proteins important for virus replication through the activities of VP16 and ICP0. In contrast, the only viral gene expressed at high levels in latently infected neurons is the gene specifying the latency-associated transcripts (LATs), which are not known to encode proteins and whose functions are unknown. Thus, all known virally-encoded transactivators are absent. In addition, many of the cell cycle-associated proteins present in cycling cells are absent in neurons. Therefore, latent viral genomes are dependent upon cellular factors associated with the neuronal stress response to induce reactivation.

Role of ICP0 in Productive Infection

Although not essential for virus replication, ICP0 is necessary for efficient replication at low multiplicities of infection (MOI). ICP0-null mutant virus particles enter cells and induce ICP4 transcription as efficiently as wild type virus; however, most genomes are replication-incompetent, and productive infection stalls. Although ICP0 is a global transcriptional activator of many cellular and viral genes, it does not function as a typical DNA-binding protein. The mechanisms by which it activates transcription are unclear, though it appears to act before transcript initiation.

Role of ICP0 in Reactivation from Latency

In addition to its roles in facilitating productive infection, ICP0 is necessary and sufficient for efficient reactivation from latency. Relative to wild type virus, ICP0-null mutant viruses used to establish latency in neurons at genome levels equivalent to those of wild type virus were unable to reactivate efficiently from trigeminal ganglia (TG) following explant and heat shock. When ICP0 was provided in trans, reactivation reached wild type levels.

Stresses that Induce Reactivation from Latency also Complement the Growth Deficiencies of ICP0— Viruses and Induce Expression of ICP0 and Other Immediate-Early Viral Regulatory Proteins

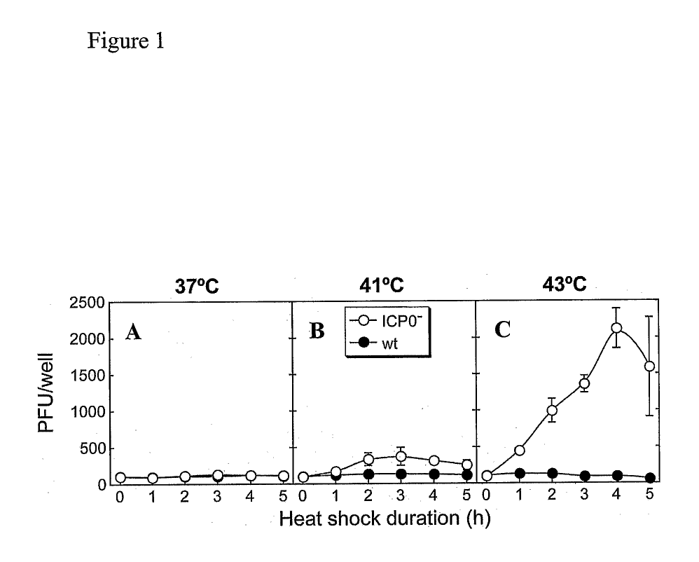

We have found that two stresses that can induce reactivation of HSV from latency also complement the deficiency in the plating and replication efficiencies for ICP0⁻ viruses. The first stress we tested was heat shock (which is analogous to fever during viral infection). In one experiment, 24-h-old monolayers were incubated 0, 1, 2, 3, 4, or 5 h at 37° C., 41° C., or 43° C. prior to infection. Immediately after incubation, monolayers were infected with 100 PFU/dish wild type (KOS) or 5-100 PFU ICP0-virus (n212) (FIG. 1). The number of plaques was multiplied by 1-20 to normalize to 100 PFU/dish. Symbols represent the average number of plaques/dish and the error bars indicate the standard deviation of 2-4 independent measurements. The plating efficiency of the ICP0⁻ virus was enhanced >25 fold when cells were heat shocked for 4 h at 43° C. prior to infection. The replication efficiency of an ICP0⁻ virus is ˜25-lower than for wild type virus, so the plating efficiency after 4 hours of heat shock at 43° C. is approximately the same as wild type.

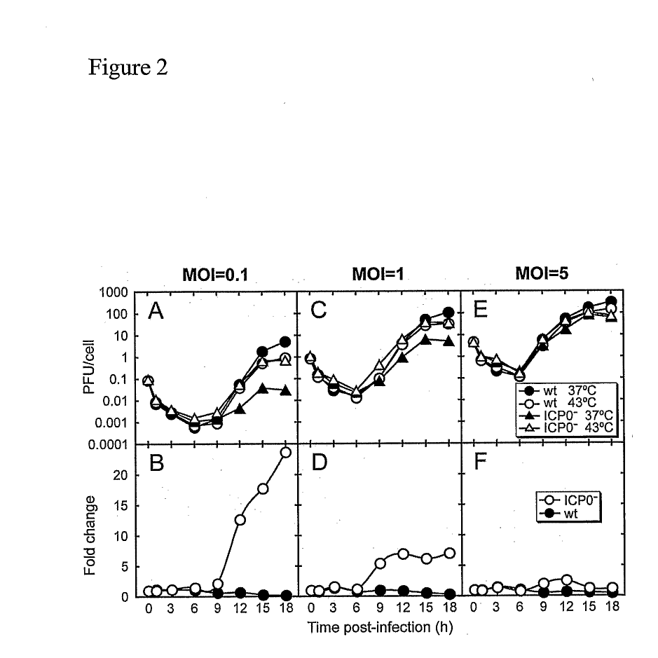

After observing that the plating efficiency of ICP0⁻ viruses is enhanced by the application of heat shock to cells prior to infection (FIGS. 1A-1C), we asked if replication efficiency at low MOIs would be similarly enhanced. Monolayers of 24 hour-old Vero cells were incubated at 37° C. or 43° C. for 4 hours and infected for 1 hour with either wild-type or an ICP0⁻ virus (n212) at MOIs of 0.1, 1, or 5 (FIG. 2A-F). Viral titers for wild-type and ICP0⁻ viruses were determined on Vero and L7 cells, respectively. Infected cells were incubated at 37° C. and harvested at 3-hour intervals. Virus titers were determined by standard plaque assay, with wild-type virus titrated on Vero cells and ICP0⁻ virus titrated on ICP0-expressing L7 cells. The titers of the inocula were also determined by standard plaque assay and are represented as the 0-h time points. FIGS. 2A, C, and E show virus replication as PFU/cell, and FIGS. 2B, D, and F show the same data plotted as fold change (PFU/cell for heat-shocked cells divided by PFU/cell for non-heat-shocked cells). On non-heat-shocked monolayers the MOI-dependent replication defect of the ICP0⁻ virus relative to wild type at 18 hours post-infection (FIG. 2A, C, and E, solid symbols) was similar to that reported previously (Cai and Schaffer J. Virol. 63:4579-4589 (1989), Everett et al., J. Virol. 78:1763-1774 (2004), and Sacks et al., J. Virol. 61:829-839 (1987)). At an MOI of 0.1 PFU/cell (FIGS. 2A and B), the amount of infectious virus present between 1 and 9 hours post-infection was similar for both viruses in heat-shocked and non-heat-shocked cells. From 12 to 18 h post-infection the amount of infectious ICP0⁻ virus in non-heat-shocked cells was reduced ˜150-fold relative to wild type. In heat-shocked cells wild-type replication was decreased ˜6-fold, whereas ICP0⁻ virus replication was enhanced nearly 25-fold, such that the two viruses exhibited similar replication efficiencies. At an MOI of 1, the pattern of virus production was similar to that observed at the lower MOI, except that the replication defect of the ICP0 virus was less severe. Heat shock produced enhancement of ˜5-fold for the ICP0⁻ virus (FIGS. 2C and D) such that the two viruses exhibited similar replication efficiencies. At high MOIs (>˜5), ICP0⁻ viruses replicate as well as wild-type virus. Accordingly, at an MOI of 5 little or no enhancement in replication of the ICP0⁻ virus occurred following heat shock (FIGS. 2E and F). These results demonstrate that the replication defect of an ICP0⁻ virus at low MOIs can be entirely overcome by subjecting cells to heat shock prior to infection.

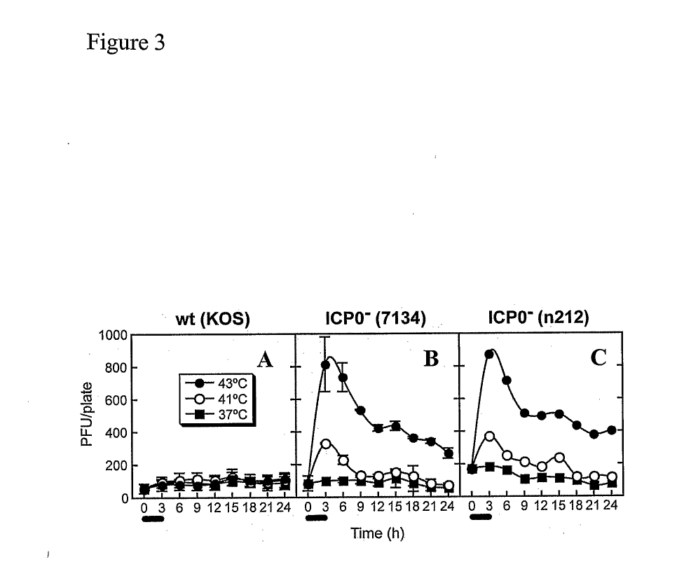

Heat shock induces the expression/activation (or repression/inactivation) of certain cellular activities resulting in enhanced plating and replication efficiencies of ICP0⁻ viruses. To better define the nature of the alteration in cellular activities responsible for the enhanced plating and replication efficiencies, we examined the kinetics of the decline of plating efficiency of ICP0⁻ viruses following heat shock at 41° C. or 43° C. For this purpose 24-hour-old Vero cell monolayers were incubated at 37° C., 41° C., or 43° C. for 3 hours followed by incubation at 37° C. for up to 21 hours (FIG. 3). At 3-h intervals, monolayers were infected with approximately 100 PFU/plate wild-type or approximately 20 or 100 PFU/plate ICP0⁻ viruses to determine plating efficiency. If monolayers infected with 100 PFU/plate ICP0 virus resulted in too many plaques to count, those infected with 20 PFU were counted instead, and the result was multiplied by 5. Infected cells were incubated at 37° C. for 4 days, stained, and plaques counted. The plating efficiency of wild-type virus was not significantly altered by any treatment. In contrast, following 3-hour incubation at 41° C. and 43° C., plating efficiencies of the ICP0⁻ viruses peaked at 3- and 9-fold, respectively, at the time of removal from heat stress. For monolayers incubated at 41° C., the plating efficiencies of ICP0⁻ viruses declined for 6 hours after removal of the stress (t=9 hours on the x-axis of FIG. 3) until similar to the plating efficiency on non-heat-shocked monolayers. For monolayers incubated at 43° C., the enhanced plating efficiency declined for 6 h after removal of the stress at approximately the same rate as for monolayers incubated at 41° C. After 6 hours, however, the plating efficiency declined at a reduced rate and remained detectable throughout the duration of the experiment. In summary, the heat-shock-inducible cellular factors that are responsible for complementation of the ICP0⁻ viruses are expressed transiently after removal of the heat stress.

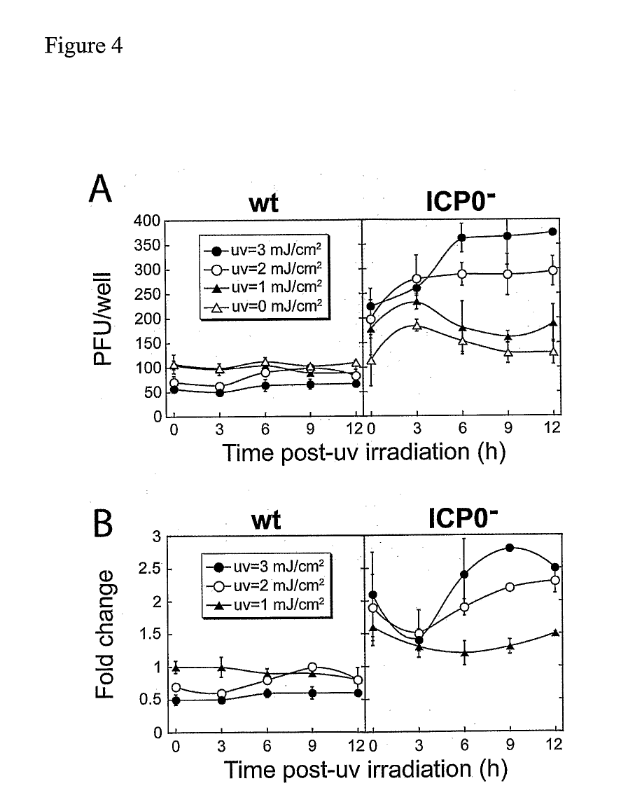

The second stress we tested was UV irradiation (analogous to sunburn) (FIGS. 4A-B). 24-hour-old monolayers of Vero cells were washed once with Hanks' Balanced Salt Solution (HBSS) and overlaid with 1 ml of HBSS. Cells were then subjected to 0, 1, 2, 3, or 4 mJ/cm² UV-C irradiation. 4 mJ/cm² was determined to be a lethal dose. HBSS was immediately replaced with normal medium, and cells were incubated at 37° C. until the time of infection (shown on the x-axis). Monolayers were infected at 3-hour intervals with 100 PFU/dish wt (KOS) or ICP0⁻ virus (n212). Symbols represent the average number of plaques/dish, and the error bars indicate the standard deviation of 4 independent measurements. As the dose of UV irradiation increased, the plating efficiency of the ICP0⁻ virus was enhanced. As was the case following heat shock, greater stress resulted in greater enhancement of plating efficiency.

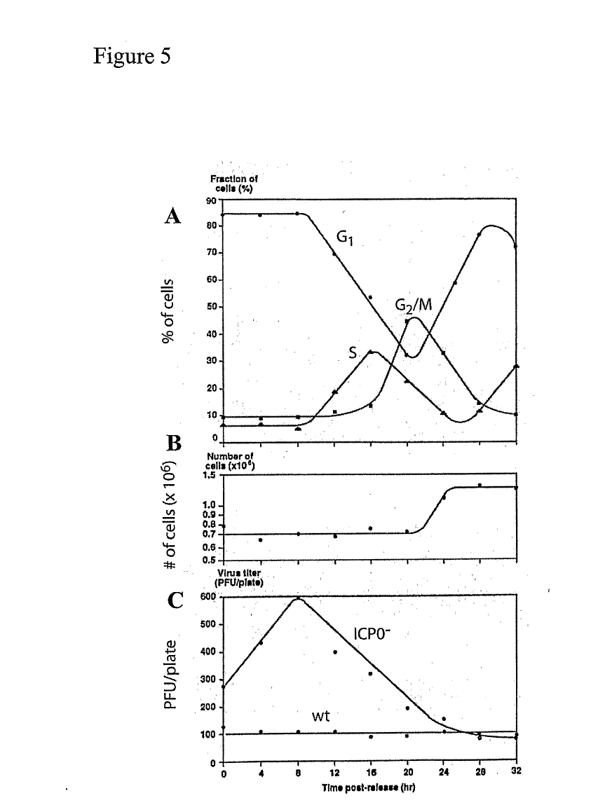

An additional cellular treatment that resulted in enhanced efficiency for an ICP0⁻ virus was isoleucine (Ile) deprivation/refeeding. Cai and Schaffer (J. Virol. 65:4078-4090, 1991; incorporated by reference in its entirety; in particular, see FIG. 5 and page 4083) synchronized monolayers of 24-h-old Vero cells (immortalized African green monkey kidney cells) in G₀/G₁ phase by incubation in Ile-free medium for 42 hours followed by refeeding with Ile-containing medium and infected with either wt (KOS) or ICP0⁻ (7134) virus every 4 hours after release of the block. The number of plaques produced by the ICP0⁻ virus increased 6-fold during the first 8 h then decreased through the next 18 hours post-release of the Ile block. In contrast to the ICP0⁻ virus, the wild type virus produced the same number of plaques at all time points tested. Replicate cell monolayers at each time point were also stained with propidium iodide and analyzed with a fluorescence activated cell sorter (FACS). This analysis determines the amount of DNA per cell, which corresponds with the stage of the cell cycle. Because the peak in plating efficiency occurs at the same time as cells transition between G₁ and S phase, it was initially thought that the ICP0⁻ virus was able to plate more efficiently at this stage of the cell cycle. As we will discuss below, a stress response caused by glutamine (Gln) deprivation/refeeding rather than Ile deprivation/refeeding or a specific stage of the cell cycle likely produced the enhanced ICP0⁻ virus plating efficiency seen in this experiment.

We have also demonstrated that Ile deprivation and refeeding induces expression of the ICP0 promoter as well as other viral promoters. Vero cells and NB41A3 cells (mouse neuroblastoma cells) were treated by Ile deprivation/refeeding as in the previous experiment. The expression of viral gene promoters was then analyzed. Promoter-chloramphenicol acetyltransferase (CAT) expression from plasmids was measured following transfection of Vero cells treated by Ile-deprivation/refeeding. ICP0 and ICP4 promoter-CAT expression increased 6- or 4-fold, respectively, at early times (0-6 hours post-release) then decreased through 10 hours post-release. In cells from the mouse neuroblastoma cell line, NB41A3, ICP0, ICP4, and ICP22/47 promoter-CAT expression was increased 6-, 10-, or 5-fold, respectively.

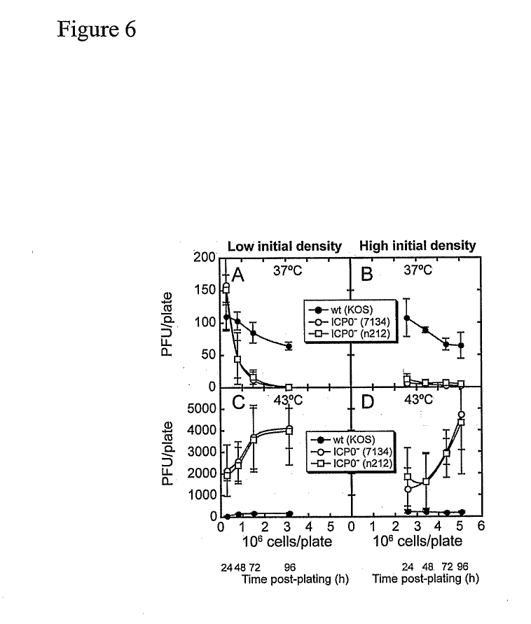

We also sought to determine whether the age or the density of the monolayer is the factor that results in reduced plating efficiency, and if these aged/dense monolayers can be induced by heat shock to support efficient ICP0⁻ virus plaque formation. At 24-hour intervals post-seeding at low (1.5×10⁵ cells/35-mm plate, FIGS. 6A and 6C) or high (1.5×10⁶ cells/35-mm plate, FIGS. 6B and 6D) initial density, Vero cell monolayers were incubated at 37° C. or heat shocked at 43° C. for 3 hours. Cells were either harvested and counted, or replicate monolayers were infected with wild-type (KOS) or ICP0⁻ viruses (7134 or n212) at the times indicated and incubated at 37° C. for 4 days. Plaque counts for infections of 5 PFU/plate were multiplied by 20 to normalize to an input of 100 PFU/plate.

On monolayers seeded at low initial density, we confirmed the previously described decrease in ICP0⁻ virus plating efficiency resulting in the nearly complete absence of plaques on monolayers incubated continuously at 37° C. for 96 hours (FIG. 6A). In contrast, wild-type virus showed only a moderate decrease (˜2-fold) in plating efficiency on similar monolayers. On monolayers seeded at high initial density the decrease in wild type plating efficiency was similar to that observed at low initial density (FIG. 6B). For the ICP0⁻ viruses, however, very few plaques formed at any time post-plating. A comparison of the plating efficiencies of the ICP0⁻ viruses at similar densities [2.5×10⁶ cells, near 96 hours post-plating for low initial density (FIG. 6A) and 24 hours post-plating for high initial density (FIG. 6B)] revealed that very few plaques formed on monolayers at either initial density. In contrast, a comparison of the plating efficiencies of the ICP0⁻ viruses at 24 hours post-plating revealed that at low initial density 150 plaques formed, whereas at high initial density very few plaques formed. These observations suggest that cell density rather than age of the monolayer is the predominant factor underlying the reduced plating efficiency of ICP0⁻ viruses.

As described above, monolayers seeded at low or high initial densities were heat shocked or remained at 37° C. for 3 hours and were infected with wild-type or ICP0⁻ viruses (FIGS. 6C and D). Heat shock prior to infection did not significantly alter the plating efficiency of wild-type virus whereas the plating efficiency of the ICP0-viruses was greatly enhanced, with the maximum change being an increase from 0 to >4000 plaques at 96 hours post-plating for monolayers seeded at both initial densities (FIGS. 6C and D). This observation suggests that the decrease in ICP0⁻ virus plating efficiency on dense monolayers is likely due to the loss of heat shock-inducible cellular activities present in low-density, but not high-density monolayers (or, conversely, heat shock-labile/repressible activities that inhibit plaque formation by ICP0⁻ viruses and accumulate in high-density monolayers). In summary, as monolayers age and become more dense, the decrease in plating efficiency of ICP0⁻ viruses is due to increased cell density, and this effect can be overcome by cellular heat shock prior to infection.

Glutamine Deprivation/Refeeding rather than Isoleucine Deprivation/Refeeding Results in Enhanced Plating Efficiency of an ICP0⁻ Virus

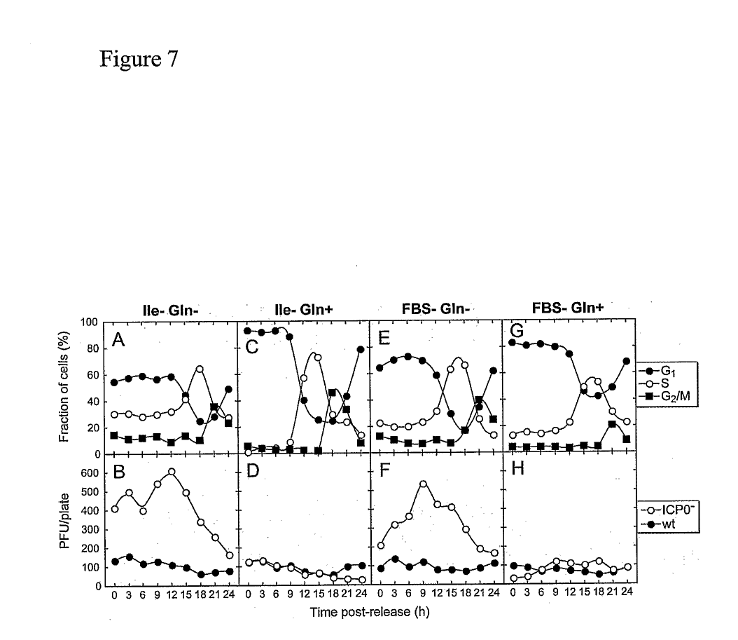

While attempting to repeat the Cai and Schaffer experiment shown in FIG. 5, we found that we could only replicate the results in the absence of glutamine. We incubated 24-h-old Vero cell monolayers for 42 h in Ile-free media containing 0 or 2 mM glutamine or serum-free media containing 0 or 2 mM glutamine. Like isoleucine deprivation, serum starvation is a method commonly used to synchronize cells to G₁ phase of the cell cycle. We then replaced the media with normal growth medium, infected cells with wild type (KOS) or ICP0⁻ virus (n212), and analyzed cells for cellular DNA content at 3-h intervals (FIG. 7). We found that while all growth conditions resulted in cell synchrony, only incubations in the absence of glutamine resulted in enhanced plating efficiency of the ICP0⁻ virus. Glutamine is widely known to be unstable in growth media. For the experiment shown in FIG. 5, Cai and Schaffer reported that the isoleucine-free medium contained 2 mM glutamine, but based on the results shown in FIG. 4, it is likely that most of the glutamine had degraded.

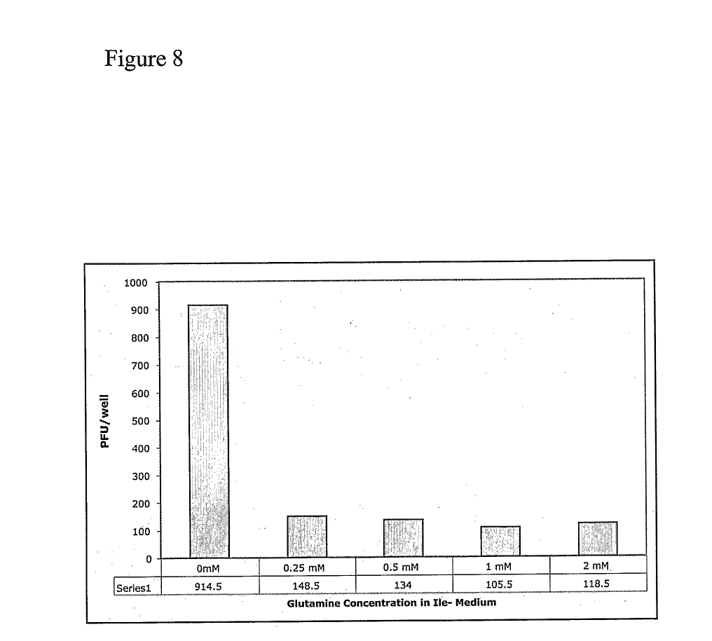

To determine the amount of glutamine necessary to prevent the enhanced plating efficiency of an ICP0⁻ virus, we incubated 24-h-old Vero cell monolayers in isoleucine-free media containing 0, 0.25, 0.5, 1, or 2 mM glutamine for 42 h then replaced the media with normal growth medium. We infected the monolayers with an ICP0⁻ virus (n212) 9 h after the addition of normal medium (FIG. 8). In a similar experiment, 24-h-old Vero cells were incubated 24, 48, or 72 hours in the absence of glutamine, refed with normal medium and infected with an ICP0⁻ virus (n212) 9 h later. The 48-h incubation enhanced the plating efficiency ˜6-fold while the 24-h incubation enhanced the plating efficiency ˜2-fold. After 72 hours without glutamine the cells were unable to survive. Like heat shock and UV irradiation, greater stress resulted in greater enhancement of the plating efficiency of the ICP0⁻ virus.

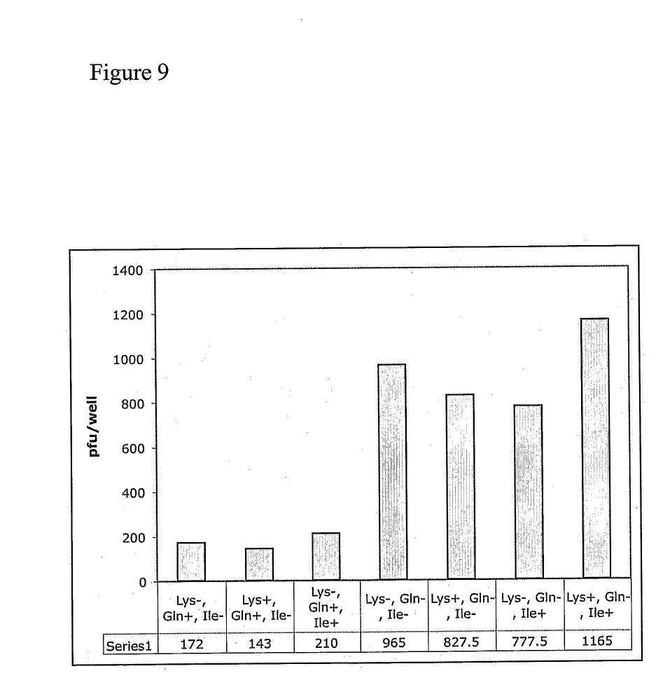

The presence of the amino acid L-lysine has been reported to decrease the replication efficiency of HSV in cell culture and is commonly used therapeutically to prevent recurrent HSV-1 and -2 lesions (see, e.g., U.S. Pat. Nos. 6,231,889, 6,455,061, and 6,632,445, and Singh et al., Altern. Med. Rev. 10:123-127, 2005). We performed the experiment shown in FIG. 9 to determine if lysine deprivation/refeeding alone or in combination with isoleucine and/or glutamine deprivation/refeeding could enhance the plating efficiency of an ICP0⁻ virus. Twenty-four-h-old Vero cell monolayers were incubated 42 h in media lacking isoleucine, glutamine, or lysine in all combinations. Media were replaced with normal growth medium, and after a 9-h incubation, monolayers were infected with ICP0⁻ (n212). Glutamine deprivation/refeeding caused an ˜9-fold enhancement regardless of which other amino acids were absent. Lysine deprivation/refeeding caused a slight increase. In a separate experiment we found that lysine deprivation/refeeding enhanced the plating efficiency of the ICP0⁻ virus by ˜2-3 fold. Isoleucine had little or no effect.

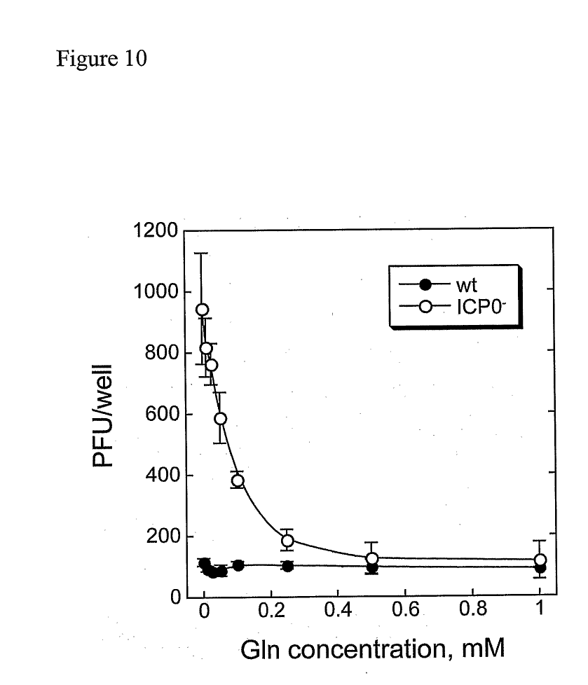

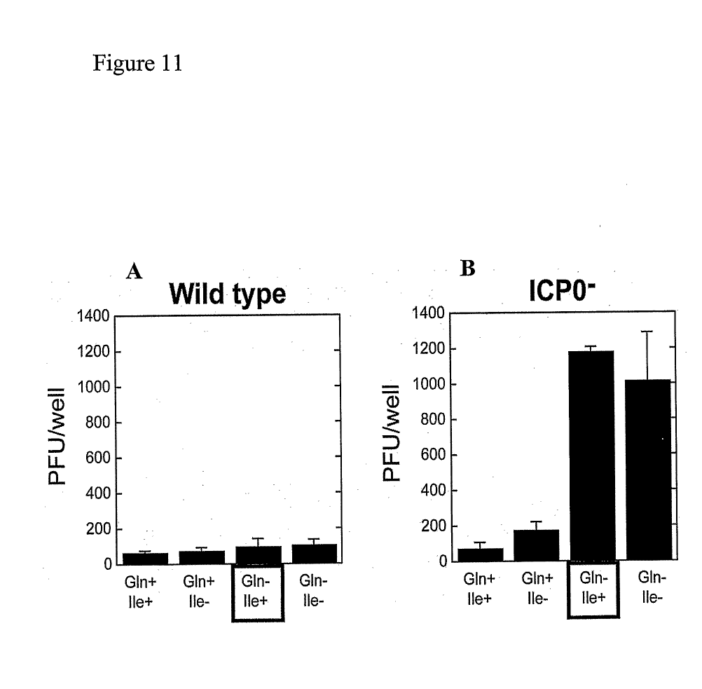

In a separate experiment shown in FIG. 10, monolayers of cells were incubated for 42 hours in Ile-free medium containing 0-1 mM glutamine and then in normal medium for 9 hours prior to infection. Cells were then infected with wild type or ICPO-virus to determine plating efficiency as described herein. The enhanced plating efficiency was seen only when the glutamine concentration was less than 0.5 mM. The reported human serum glutamine concentration is approximately 0.5 mM. The concentration of 2 mM glutamine reported by Cai and Schaffer, supra, was probably in error and was more likely less than 0.1 mM glutamine. In order to determine the role of isoleucine in these experiments, monolayers of cells were incubated for 42 hours in normal medium (Gln+ Ile+) or media lacking Ile, Gln, or both. Cells were then infected with wild type or ICP0⁻ virus to determine plating efficiency as described herein. As shown in FIG. 11, the media lacking only glutamine produced results similar to the media lacking both glutamine and isoleucine indicating that the enhanced ICP0-virus plating efficiency is due to glutamine deprivation rather than Ile deprivation.

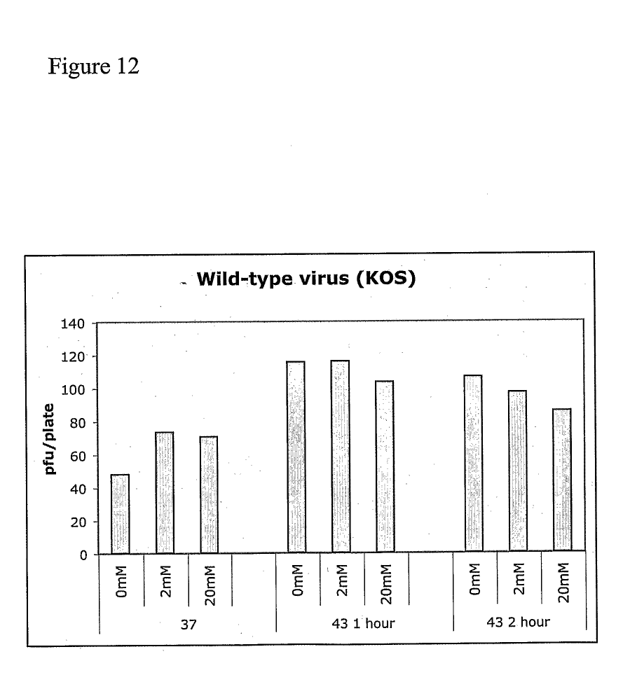

To determine if the presence of increased glutamine levels could decrease the plating efficiency of a wild type virus on heat shocked cells (see FIG. 1), Vero cell monolayers were incubated in media containing 0, 2, or 20 mM glutamine for 24 h (normal medium contains 2 to 6 mM glutamine), then incubated 1 or 2 h at 43° C. or left at 37° C. for 2 additional hours (see FIG. 12). The plating efficiency of wild type virus on heat-shocked cells was slightly reduced in cells incubated in 20-mM glutamine.

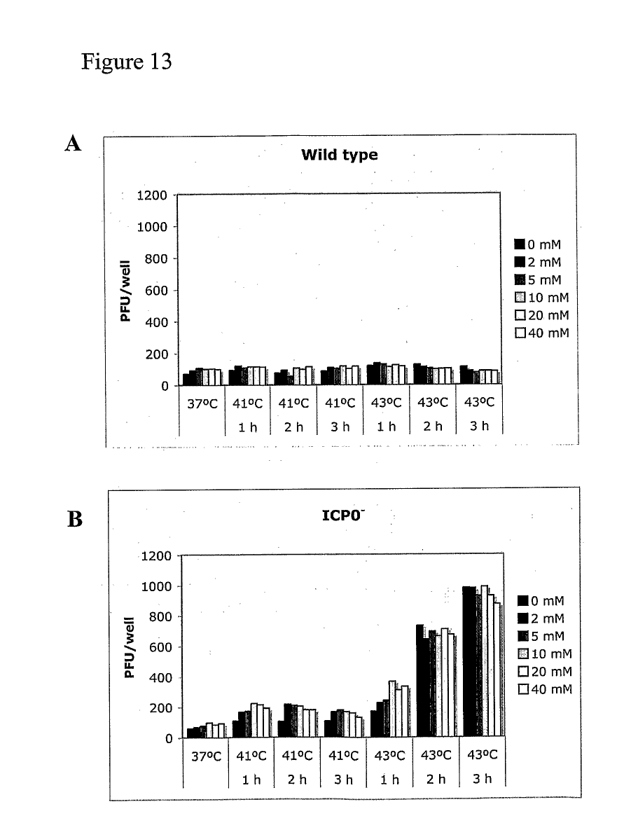

In a separate experiment shown in FIG. 13, high glutamine concentration did not prevent the enhanced plating efficiency of the ICP0-virus due to heat shock. For this experiment, monolayers were incubated for 24 hours at 37° C. in the presence of 0-40 mM glutamine and then heat shocked at 41° C. or 43° C. for 1, 2, or 3 hours. Cells were then infected with wild type or ICP0⁻ virus to determine plating efficiency as described herein. As shown in FIG. 13, high glutamine concentration did not overcome the effects of heat shock on the plating efficiency of the ICP0 virus.

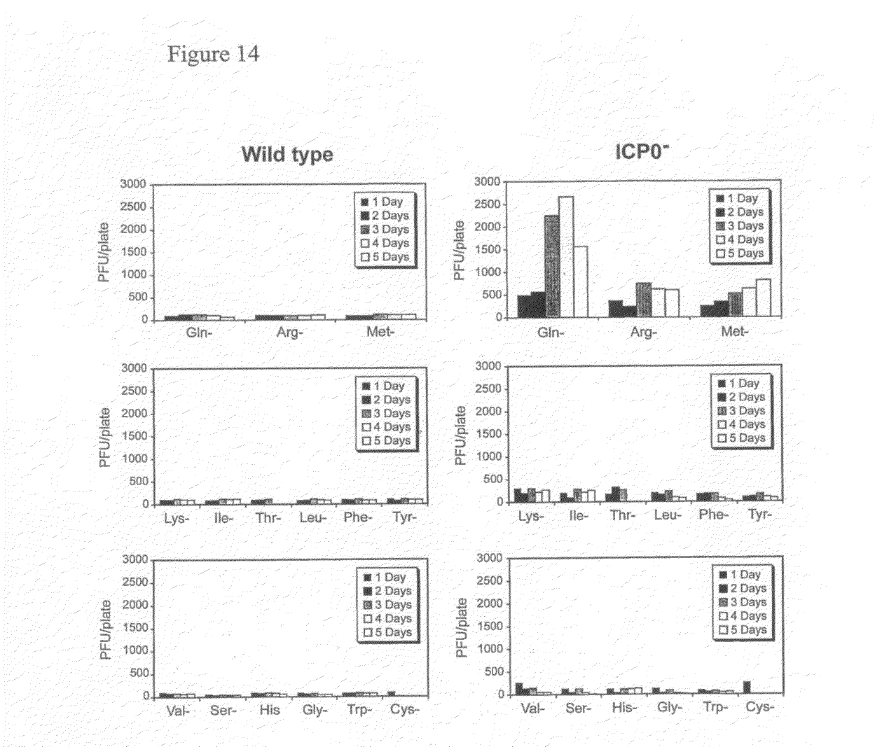

Given Cai and Schaffer's (supra) results showing that treating cells with isoleucine deprivation/refeeding prior to infection resulted in enhanced plating efficiency for an ICP0⁻ virus, we sought to determine if any of the other essential amino acids had a similar effect on plating efficiency. For these experiments, monolayers were incubated for 1-5 days in the absence of each of the 15 amino acids present in DMEM. Cells were then infected with wild type or ICPO-virus to determine plating efficiency as described herein. As shown in FIG. 14, glutamine deprivation and to a lesser extent arginine and methionine deprivation resulting in enhanced plating efficiency of the ICP0-virus. Deprivation of the other 12 amino acids had little effect.

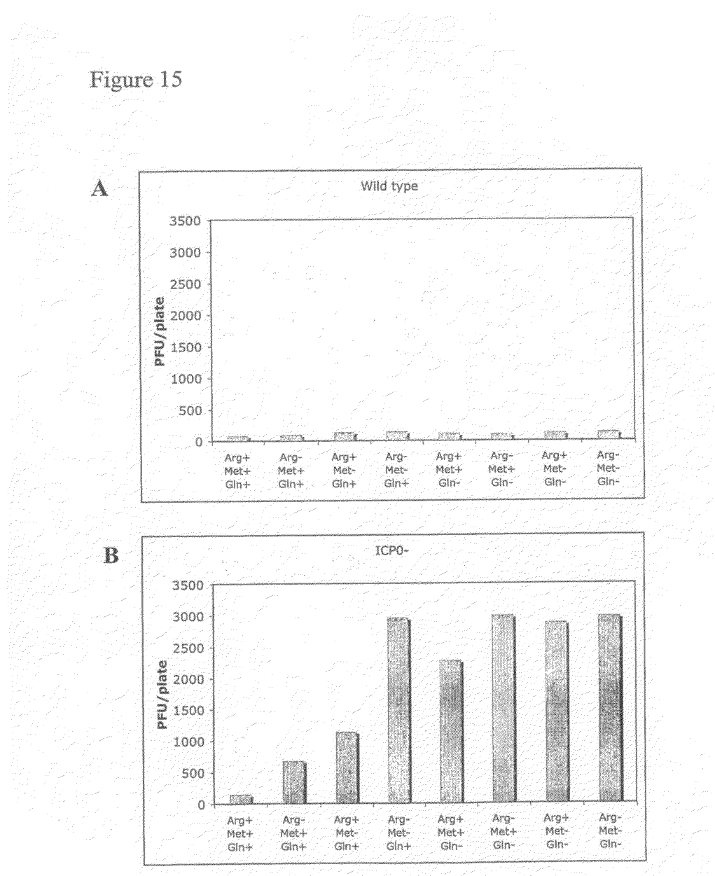

In order to further understand the effects of arginine and methionine on the plating efficiency, monolayers of cells were incubated for three days in media containing all combinations of arginine, methionine, and glutamine. Cells were then infected with wild type or ICPO-virus to determine plating efficiency as described herein. As shown in FIG. 15, there is a synergistic effect for deprivation of arginine and methionine that enhances the plating efficiency at least as much as glutamine deprivation and possibly slightly more. These results indicate that the absence of arginine and methionine together can act to enhance the plating efficiency of the virus and may be involved in reactivation from latency.

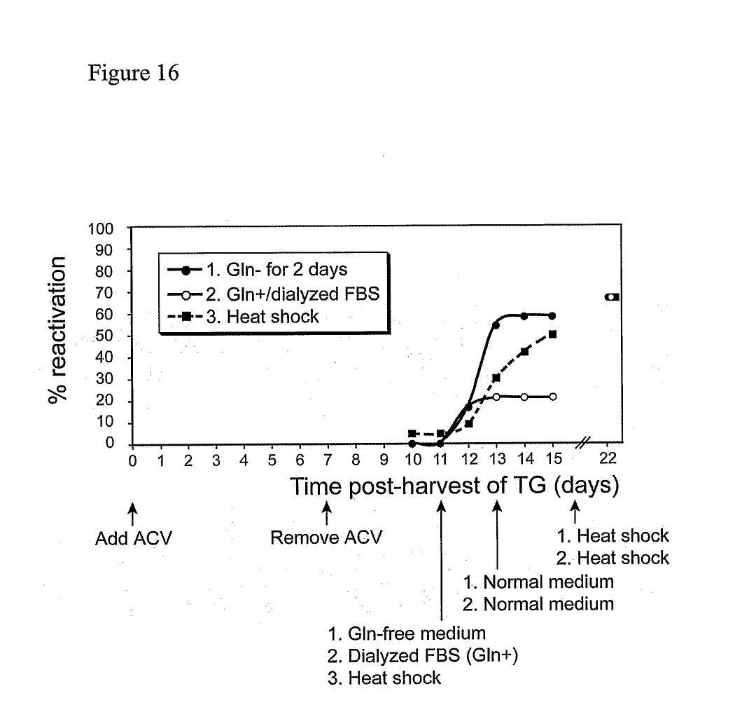

A mouse ocular/TG cell culture model was used to determine if glutamine deprivation induces reactivation of wild-type virus from latency. (For details on the model, see, for example, Leib et al., J. Virol. 63:759-768 (1989), Halford et al., J. Virol. 74:5957-5967. (2000), and Halford et al., J. Virol. 75:3240-3249 (2001)). For this experiment, mice were infected with the wild-type virus and 30 days later the trigeminal ganglia (TG) were removed and incubated in 3 24-well plates in medium containing acyclovir (ACV). As indicated below the x-axis of FIG. 16, on day 7 the acyclovir was removed. On day 11, 1 24-well plate was treated by: washing once with HBSS and replacing with Gln-free medium supplemented with 10% dialyzed FBS (red); washing once with HBSS and replacing with Gln-containing medium supplemented with 10% dialyzed FBS (green); or 3-hour incubation at 43° C. followed by replacement of medium with fresh medium (blue). On day 13, Gln-free and Gln-containing medium with dialyzed FBS was replaced with medium containing Gln and 10% normal FBS. On day 16, the 2 plates not previously heat shocked were incubated for 3 hours at 43° C. followed by replacement of medium with fresh medium. Starting on day 10, 10% of the volume of each well was transferred daily to 1 well of a 96-well plate previously seeded with Vero cells. After incubation at 37° C. for 7 days, monolayers of Vero cells in the 96-well plates were examined by light microscopy for cytopathic effect. The y-axis above indicates the percentage of wells for each treatment (n=24) positive for cytopathic effect. These results demonstrate that TG incubated in 0 mM Gln reactivated more rapidly and to the same extent as heat-shocked TG. TG incubated in the presence of 2 mM Gln exhibited significantly reduced reactivation.

The following materials and methods were used in the experiments described above.

Cells and viruses. Vero cells, derived from African green monkey kidney cells (CCC-81, American Type Culture Collection, Manassas, Va.) and L7 cells, a derivative of Vero cells stably transformed with ICP0 (Samaniego et al., J. Virol. 71:4614-4625 (1997)), were propagated in Dulbecco's modified Eagle's medium (DMEM) with high glucose (4500 mg/L), 4 mM glutamine (Gln), and no sodium pyruvate (catalog #11965, Invitrogen, Carlsbad, Calif.) supplemented with 10% fetal bovine serum (FBS) (Biomeda, Foster City, Calif.), 2 mM Gln (6 mM final concentration), penicillin G (100 units/ml), and streptomycin (100 μg/ml). This medium was used for all experiments except the Ile deprivation and serum starvation experiments. Vero cells were passaged at least twice per week to a maximum of 25 passages after receipt from ATCC. Cells were incubated at 37° C. in a humidified incubator with 5% CO₂.

HSV-1 strain KOS was the wild-type virus used in all experiments. KOS-derived ICP0⁻ viruses included 7134, containing lacZ in place of ICP0 (Cai and Schaffer 1989 supra), and n212, containing nonsense mutations in all three reading frames at nucleotide 212 (Cai and Schaffer 1989 supra and Cai et al., J. Virol. 67:7501-7512 (1993)).

The KOS strain of HSV-1 is propagated in Vero cells (ATCC No. CCL-81, American Type Culture Collection, Manassass, Va.), titered by viral plaque assay, and stored at −70° C. until used to infect animals.

Ocular swabs are immersed in 0.2 ml of complete tissue culture medium consisting of RPMI-1640, 10% fetal bovine serum (FBS), and an antibiotic-antimycotic mixture (Life Technologies, Grand Island, N.Y.). The swabs in culture medium are stored at 4° C. until testing by plaque assay. The determination of infectious virus in the cornea and in the trigeminal ganglia of animals is performed in a similar manner. Pairs of corneas and ganglia from individual mice are placed in separate tubes in 0.2 ml of complete medium, homogenized, clarified by centrifugation at 14,000×g for 5 minutes and the supernatant tested for infectious virus on Vero cells.

Cell Synchronization.

A. Ile deprivation and serum starvation. DMEM was prepared by dissolving all components individually, except Ile and Gln, in water according to Invitrogen's formulation for DMEM (catalog #11965) with 3 substitutions: 200 mg/l calcium chloride (anhydrous) for 264.92 mg/l calcium chloride dihydrate, 48 mg/l L-cystine for 63 mg/l L-cystine dihydrochloride, and 72 mg/l L-tyrosine for 104 mg/l L-tyrosine disodium salt, dihydrate. Medium was sterilized by passage through a 0.22 μm filter. Ile and Gln were added as needed, not more than two days prior to use, at final concentrations of 0.802 or 2 mM, respectively.

Sixty-mm petri plates were seeded with 5×10⁵ cells/plate and incubated 24 h at 37° C. For Ile deprivation experiments, medium was removed and monolayers were washed with warm Hanks' Balanced Salt Solution (HBSS) (37° C.) and overlaid with 4 ml warm Ile-free DMEM with 10% dialyzed FBS and 0 or 2 mM Gln. For serum starvation experiments, medium was removed and monolayers were washed with warm HBSS and overlaid with 4 ml warm serum-free DMEM with 0.802 mM Ile and 0 or 2 mM Gln. Monolayers in Ile- or serum-free media were incubated 42 h at 37° C. Media were replaced with warm DMEM containing 10% non-dialyzed FBS, 0.802 Ile, and 2 mM Gln. At 3-h intervals monolayers were infected with wild-type or ICP0-viruses to determine plating efficiency or analyzed for cellular DNA content by flow cytometry as described below.

B. Double thymidine block. Sixty-mm petri plates were seeded with 5×10⁵ cells/plate and incubated at 37° C. for 24 h. Thymidine was added to the medium to a final concentration of 2 mM, and cells were incubated for 10 h at 37° C. Thymidine-containing medium was removed, monolayers were washed 3 times with warm HBSS, and 4 ml warm thymidine-free medium was added to each plate. After incubation at 37° C. for 14 h, thymidine was again added to the medium at a final concentration of 2 mM, and cells were incubated at 37° C. for 10 h. Thymidine-containing medium was removed, monolayers were washed 3 times with warm HBSS, and 4 ml warm thymidine-free medium was added to each plate. At 3-h intervals monolayers were infected with wild-type or ICP0⁻ viruses to determine plating efficiency or analyzed for DNA content by flow cytometry as described below.

Flow cytometry to determine cellular DNA content. Cells in 60-mm petri plates were harvested by trypsinization, pelleted, and resuspended in 300 μl of cold phosphate-buffered saline (PBS) and transferred to a microcentrifuge tube containing 700 μl of cold ethanol. Cells were stored at 4° C. until analyzed (less than 7 days). Cells were pelleted, washed once with PBS, and resuspended in 500 μl propidium iodide solution consisting of 50 μg/ml propidium iodide, 0.58 mg/ml sodium chloride, 1.065 mg/ml sodium citrate dihydrate, 0.06% NP40, and 0.4 mg/ml RNase A. Stained cells were analyzed with a FACSCaliber flow cytometer (BD Biosciences, San Jose, Calif.). The percentage of cells in each phase of the cell cycle was determined using the ModFit LT software package (Verity Software House, Inc., Topsham, Me.).

Heat shock and cold shock. Thirty-five-mm petri plates were seeded with 1.5×10⁵ Vero cells/plate and incubated at 37° C. for 24 h. For heat shock, monolayers were transferred to incubators set to 37° C., 41° C., 43° C., or 44° C. and containing 5% CO₂. For cold shock, monolayers were transferred to incubators set to 34° C. or 37° C. and containing 5% CO₂, or the plates were sealed with Parafilm (Pechiney Plastic Packaging, Inc, Chicago, Ill.) and incubated at 4° C., 22.5° C., or 37° C. in atmospheric CO₂. Monolayers were incubated for 1 to 5 h and infected with wild-type or ICP0⁻ viruses to determine plating efficiency as described below.

UV-C irradiation. Six-well plates (35-mm wells) were seeded with 1.5×10⁵ Vero cells/well and incubated 24 h at 37° C. Medium was aspirated in groups of 6 plates for individual time points, cells were washed once with 2 ml/well warm PBS (37° C.). PBS was aspirated, and 2 ml warm PBS was added to each well. Plates were placed in a UV crosslinker (UV Stratalinker 1800, Stratagene, La Jolla, Calif.) and exposed to 0, 1, 2, 3, 4, or 6 mJ/cm² UV-C irradiation. For all time points except 0 h post-irradiation, PBS was replaced with warm medium, and cells were incubated at 37° C. for 3, 6, 9, or 12 h prior to infection with wild-type or ICP0⁻ viruses to determine plating efficiency. For the O-h time point, PBS was aspirated, and monolayers were infected immediately.

Infections to determine plating efficiency. Vero cell monolayers in 35-, 60-, and 100-mm plates were infected with volumes of 200 μl, 400 μl, or 2 ml/plate, respectively, with 5, 10, 20, 40, or 100 PFU/plate. For all experiments except Ile deprivation and serum starvation, titers were determined on 24-h-old Vero cell monolayers that had been seeded at a concentration of 1.5×10⁵ Vero cells/35-mm plate and incubated at 37° C. For Ile deprivation and serum starvation, a higher titer of ICP0⁻ virus was used such that 100 plaques were produced at the O-h time point for Ile deprivation/refeeding with 2 mM Gln (see FIG. 2F). Plates were incubated at 37° C. and shaken every 15 minutes for 1 h. Two, 4, or 10 ml of DMEM containing 5% FBS and 0.5% methyl cellulose was added to 35-, 60-, and 100-mm plates, respectively, and monolayers were incubated 3-5 days at 37° C. to allow plaques to form. Medium was aspirated, cells were stained with crystal violet, and plaques were counted. The number of plaques was normalized to 100 PFU by multiplying the resulting number of plaques by 20, 10, 5, or 2.5 for infections of 5, 10, 20, or 40 PFU/plate, respectively. These dilutions were necessary to allow for sufficient separation of plaques. For example, in FIG. 1, incubation of 4 h at 43° C. produced 2000 plaques of the ICP0-viruses, which are too numerous to count on a 35-mm plate. Therefore, this infection was performed by infecting with 10 PFU and multiplying the resulting ˜200 plaques by 10.

One-step growth curves to determine replication efficiency. Thirty-five-mm petri plates were seeded with 1.5×10⁵ Vero cells/plate and incubated at 37° C. for 24 h. Cells from 3 plates were harvested by trypsinization, pooled, pelleted, resuspended, and counted. MOIs were calculated for the average number of cells per plate. Monolayers were heat shocked at 43° C. for 4 h immediately prior to infection or remained at 37° C. Monolayers were infected at MOIs of 0.1, 1, and 5 PFU/cell in a volume of 100 μl/plate. Titers for KOS and n212 were determined on 24-h-old monolayers of Vero and L7 cells, respectively. Cells were incubated at 37° C. with shaking every 15 min for 1 h. Immediately following infection monolayers were washed 3 times with warm HBSS (37° C.), and 1 ml of warm medium was added to each plate. At 1 h post-infection and at 3-h intervals post-infection, 1 plate from each combination of virus (KOS or n212), incubation temperature (37° C. or 43° C.), and MOI (0.1, 1, or 5) was frozen at −80° C. After all samples were frozen, cells were thawed, removed from the plate by repeated pippeting with a micropipettor, lysed by sonication, and assayed for virus titer by standard plaque assay. KOS and n212 were assayed on Vero and L7 cells, respectively. After cells were infected, titers of the KOS and n212 inocula were verified by standard plaque assay and are represented in FIGS. 2A-F as the O-h time point.

Potential Uses

Acyclovir and related drugs are available to treat productive HSV-1 and -2 and varicella zoster virus. There is no treatment for the other five herpesviruses, and there is no treatment for the prevention of recurrence of the latent form of any of the herpesviruses.

We believe that conditions and factors that enhance the plating efficiency of an ICP0⁻ virus may also be involved in reactivation from latency. The absence of glutamine significantly enhanced the plating efficiency of the virus, and the presence of glutamine eliminated the enhancement. The same effect was seen for lysine, though the enhancement for lysine deprivation/refeeding was much lower than for glutamine. Lysine is commonly used therapeutically to prevent reactivation of HSV-1 and -2. The absence of arginine and methionine also enhanced the plating efficiency of the virus and the presence of glutamine with either arginine, methionine, or both eliminated this effect. Based on these results, we believe that therapeutic supplementation with glutamine will more effectively reduce the incidence of reactivation of HSV-1 from latency. In addition, supplementation with either arginine, methionine, or both, or combinations of either of these with glutamine will also effectively reduce the incidence of reactivation of HSV-1 from latency.

Because the presence of high levels of glutamine reduced the plating efficiency of a wild type virus on cells that were heat shocked for 1 or 2 hrs at 43° C., therapeutic use of glutamine can be used to prevent reactivation of a herpesvirus, or to reduce the severity of an established lytic herpesvirus infection upon reactivation.

Other herpesviruses, such as cytomegalovirus (CMV) and human herpesvirus 6 (HHV-6), are known to cause severe medical problems. CMV infection is the leading infectious cause of mental retardation in developed countries. HHV-6 infection can cause rejection of transplanted tissue, particularly bone marrow. There is no effective treatment for either infection. Administration of glutamine could become a routine therapeutic for the prevention of transmission of these herpesviruses, and their associated complications and outcomes.

Glutamine Derivatives, Conjugates, and Analogs

Compositions of the invention for treating or preventing reactivation of a latent herpesvirus, or a disease or condition associated with herpesvirus reactivation, include glutamine, or derivatives, conjugates, or analogs thereof. Glutamine derivatives that can be used in the methods and compositions of the invention include, e.g., the D- or L-amino acid, N-acetyl-L-glutamine, and a glutamine-containing peptide, e.g., a polyglutamine peptide (e.g., dipeptide glycyl-glutamine, L-alanyl-L-glutamine or glycyl-L-glutamine and glycyl-glycyl-L-glutamine). Dietary supplements containing or enriched in glutamine, as well as foods containing or enriched in glutamine can be administered to a subject to treat or prevent reactivation of a latent herpesvirus, or a disease or condition associated with herpesvirus reactivation, according to the methods of the invention.

Glutamate analogs that can be used in the methods and compositions of the invention include glutamines with a modifying group, such are an aldehyde or ketone, particularly chloromethyl, fluoromethyl, and trifluoromethyl ketones, sulfonium salts, nitriles, diazoketones, diazomethylketones, hydroxamates, alkenes, and alkynes. Glutamine derivatives that can be used in the methods and compositions also include acylated glutamine ester derivatives, such as those described in U.S. Pat. No. 5,190,782, the glutamine derivates and salts thereof described in U.S. Pat. No. 5,559,092, and the glutamine esters and ester salts described in, e.g., U.S. Pat. No. 3,979,449. Additional glutamine derivatives that can be used in the methods and compositions of the invention are disclosed in U.S. Pat. No. 5,561,111, which describes glutamine coupled with glucose and acylated glutamine derivatives having a C₂-C₆ carboxylic acid.

Pharmaceutically acceptable acid addition salts of glutamine include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihyrogenphosphate, metaphosphate, pyrophosphate, chloride, bromide, iodide, acetate, trifluoroacetate, propionate, caprylate, isobutyrate, oxalate, malonate, succinates suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like (see, for example, Berge S. M., et al., “Pharmaceutical Salts,” Journal of Pharmaceutical Science, 1977, vol. 66: 1-19).

Pharmaceutically acceptable base addition salts of glutamine are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

Certain of the glutamine compositions of the present invention can exist in unsolvated forms as well as solvated forms, including hydrated forms. In general, the solvated forms, including hydrated forms, are equivalent to unsolvated forms and are intended to be encompassed within the scope of the present invention.

Certain of the glutamine compositions of the present invention possess one or more chiral centers and each center may exist in the R(D) or S(L) configuration. The present invention includes all enantiomeric and epimeric forms as well as the appropriate mixtures thereof.

Arginine and Methionine Derivatives, Conjugates, and Analogs

Compositions of the invention for treating or preventing reactivation of a latent herpesvirus, or a disease or condition associated with herpesvirus reactivation, include arginine, methionine, arginine and methionine, or derivatives, conjugates, or analogs thereof. Arginine derivatives that can be used in the methods and compositions of the invention are known in the art and include L-arginine, precursors to L-arginine such as oligopeptides or polypeptides comprising 1-arginine, as well as foods containing or enriched in arginine. Oligopeptides of particular interest include oligopeptides of from 2 to 30, usually 2 to 20, preferably 2 to 10 amino acids, having at least 50 mol % of L-arginine, preferably at least about 75 mol % of L-arginine, more preferably having at least about 75 mol % of L-arginine. The oligopeptides can be modified by being ligated to other compounds, which can enhance absorption from the gut. Arginine or arginine derivatives, conjugates, or analogs thereof, can be administered to a subject to treat or prevent reactivation of a latent herpesvirus, or a disease or condition associated with herpesvirus reactivation, according to the methods of the invention. In one embodiment, the arginine derivative is L-arginine hydrochloride or L-arginine monohydrochloride which are non-toxic and highly soluble. Additional preferred arginine derivatives include physiological salts of arginine, such as arginine glutamate, arginine butyrate, and esters of arginine such as arginine ethyl ester (used as a nutritional supplement for improved absorption relative to L-arginine) or arginine butyl ester. Additional arginine derivates and formulations of arginine are described in U.S. Patent Application Publication Nos. 20040147567 and 200150027001.

Methionine derivatives that can be used in the methods and compositions of the invention are known in the art and include L-methionine, S-adenosyl-L-methionine (SAM), and acetyl methionine. Methionine or methionine derivatives, conjugates, or analogs thereof can be administered to a subject to treat or prevent reactivation of a latent herpesvirus, or a disease or condition associated with herpesvirus reactivation, according to the methods of the invention. Additional methionine derivatives are described in Friedman et al., J. Nutr. 118:388-97 (1988). The methionine or arginine can be administered as any physiologically acceptable salt, such as the hydrochloride salt or glutamate salt.

Combination Therapies

As shown herein, there is a synergistic effect for deprivation of arginine and methionine that enhances the plating efficiency similarly to glutamine deprivation. Therefore, the invention includes the use of arginine, methionine, glutamine, or any combination thereof. Preferably, the invention includes the use of glutamine with arginine, methionine, or both. In addition, suitable antiviral drugs effective for treating or preventing herpesvirus infection or reactivation can be prepared in combination with glutamine for use in the methods of the present invention. Suitable antiviral drugs include, e.g., urea, idoxuridine, amantadine, methisazone, cytarabine, interferons, viral thymidine kinase inhibitors, nucleosides and nucleotide analogues (e.g., acyclic nucleoside analogs, such as gancilovir, pyrophosphates, such as foscarnet, and acyclovir famciclovir, valacyclovir, penciclovir, cidofovir, adefovir, and brivudin), immune-response modifying drugs, such as resiquimod, and herpesvirus-specific vaccines. Antiviral medications are usually taken by mouth (orally), although they are sometimes given topically. In severe genital herpes outbreaks or herpes in newborns, the medications are administered intravenously (IV).

Preservatives and other additives may also be present, such as, for example, antimicrobials, anti-oxidants, chelating agents, inert gases, and the like.

Pharmaceutical Compositions

Pharmaceutical compositions of the invention containing glutamine, or derivatives, conjugates, or analogs thereof, may be administered to a mammalian subject, such as a human, directly or in combination with any pharmaceutically acceptable carrier or salt known in the art. Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. One exemplary pharmaceutically acceptable carrier is physiological saline. Other physiologically acceptable carriers and their formulations are known to one skilled in the art and described, for example, in Remington's Pharmaceutical Sciences, (18^{th} edition), ed. A. Gennaro, 1990, Mack Publishing Company, Easton, Pa.

Pharmaceutical formulations of a therapeutically effective amount of a glutamine composition of the invention, or pharmaceutically acceptable salt thereof, can be administered orally, parenterally (e.g., intramuscular, intraperitoneal, intravenous, inarterial, subcutaneous, or ocular injection, inhalation, intradermally or transdermally, optical drops, or implant), nasally, vaginally, rectally, sublingually or topically, in admixture with a pharmaceutically acceptable carrier adapted for the route of administration.

Methods well known in the art for making formulations are found, for example, in Remington's Pharmaceutical Sciences (18^{th} edition), ed. A. Gennaro, 1990, Mack Publishing Company, Easton, Pa. Glutamine compositions intended for oral use may be prepared in solid or liquid forms according to any method known to the art for the manufacture of pharmaceutical compositions. Usually, the medicament includes other materials to accelerate or otherwise promote healing. Oral compositions may additionally include zinc oxide, tannic acid, menthol, ethyl alcohol or the like. For this purpose, the zinc oxide is present in an amount from about 0.5 percent by weight to about 3 percent by weight, preferably from about 1 percent by weight to about 3 percent by weight and more preferably from about 1.5 percent by weight to about 2 percent by weight. Zinc compounds, such as zinc oxide, are included because zinc oxide functions as an astrigent, as a corrosive to promote healing, and as a mild antiseptic. Tannic acid is included because it is an astringent and precipitates protein with heavy metal ions such as zinc. Since herpesviruses contain protein components, the zinc oxide and tannic acid effectively disrupt the virus membrane. Menthol can be added to the oral formulation (for application to, e.g., the lips) because it gives a cool feeling and relieves itching. Ethyl alcohol can be included as a mild antiseptic because patients often scratch their blisters and cause secondary infection therein. The tannic acid is present in an amount from about 0.5 percent by weight to about 2 percent by weight, preferably from about 0.5 percent by weight to about 1.5 percent by weight and more preferably from about 1 percent by weight to about 1.5 percent by weight. The menthol is present in an amount from about 0.25 percent by weight to about 1 percent by weight, preferably from about 0.25 percent by weight to about 0.75 percent by weight and more preferably from about 0.30 percent by weight to about 0.50 percent by weight. The ethyl alcohol is present in an amount from about 0.30 percent by weight to about 1.0 percent by weight, preferably from about 0.30 percent by weight to about 0.75 percent by weight and more preferably from about 0.30 percent by weight to about 0.50 percent by weight.

For oral administration the glutamine compositions of the present invention may alternatively be incorporated with one or more excipients in the form of a mouthwash, dentifrice, oral spray, or sublingual orally-administered formulation. For example, a mouthwash may be prepared incorporating the glutamine composition in the required amount in an appropriate solvent, such as a sodium borate solution (Dobell's Solution). Alternatively, the glutamine composition may be incorporated into an oral solution such as one containing sodium borate, glycerin, and potassium bicarbonate, or dispersed in a dentifrice, or added in a therapeutically-effective amount to a composition that may include water, binders, abrasives, flavoring agents, foaming agents, and humectants.

When the medicament is for application to the genitals, the materials to accelerate or otherwise promote healing are the same as for oral application except that menthol and ethyl alcohol are eliminated. In this case, the zinc oxide is present in an amount from about 1 percent by weight to about 5 percent by weight, preferably from about 2 percent by weight to about 4 percent by weight and more preferably from about 2.5 percent by weight to about 3 percent by weight. The tannic acid is present in an amount from about 1 percent by weight to about 5 percent by weight, preferably from about 2 percent by weight to about 4 percent by weight and more preferably from about 3 percent by weight to about 4 percent.

The glutamine compositions may optionally contain sweetening, flavoring, coloring, perfuming, and/or preserving agents in order to provide a more palatable preparation. Solid dosage forms for oral administration include capsules, tablets, pills, powders, and granules. In such solid forms, the glutamine compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings. Solid forms of glutamine should generally be tested for stability to determine if refrigeration is needed.

Liquid dosage forms for oral administration include pharmaceutically acceptable emulsions, solutions, suspensions, syrups, and soft gelatin capsules. These forms contain inert diluents commonly used in the art, such as water or an oil medium. Besides such inert diluents, compositions can also include adjuvants, such as wetting agents, emulsifying agents, and suspending agents.

Formulations for parenteral administration include sterile aqueous or non-aqueous solutions, suspensions, or emulsions. Examples of suitable vehicles include propylene glycol, polyethylene glycol, vegetable oils, gelatin, hydrogenated napthalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the polypeptides of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the glutamine compositions. Alternatively, formulations can also be manufactured in the form of sterile, solid compositions that can be dissolved in sterile water or some other sterile injectable medium immediately before use. In general, liquid formulations of glutamine should be kept refrigerated as glutamine is heat labile in solution.

Glutamine compositions for rectal or vaginal administration are preferably suppositories that may contain, in addition to active substances, excipients such as cocoa butter or a suppository wax. Glutamine compositions for nasal or sublingual administration are also prepared with standard excipients known in the art. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops or spray, or as a gel.

The amount of active ingredient in the compositions of the invention can be varied. One skilled in the art will appreciate that the exact individual dosages may be adjusted somewhat depending upon a variety of factors, including the polypeptide being administered, the time of administration, the route of administration, the nature of the formulation, the rate of excretion, the nature of the subject's conditions, and the age, weight, health, and gender of the patient. In addition, the severity of the condition targeted by the glutamine composition will also have an impact on the dosage level. Wide variations in the needed dosage are to be expected in view of the differing efficiencies of the various routes of administration. For instance, oral administration generally would be expected to require higher dosage levels than administration by intravenous injection. Variations in these dosage levels of the glutamine composition can be adjusted using standard empirical routines for optimization, which are well known in the art. In general, the precise therapeutically effective dosage will be determined by the attending physician in consideration of the above identified factors.

The glutamine composition of the invention can be administered in a sustained release composition, such as those described in, for example, U.S. Pat. No. 5,672,659 and U.S. Pat. No. 5,595,760. The use of immediate or sustained release compositions depends on the type of condition being treated. If the condition consists of an acute or over-acute disorder, a treatment with an immediate release form will be preferred over a prolonged release composition. Alternatively, for preventative or long-term treatments, a sustained released composition will generally be preferred. Controlled delivery may be achieved by admixing the glutamine component with appropriate macromolecules, for example, polyesters, polyamino acids, polyvinyl pyrrolidone, ethylenevinylacetate, methylcellulose, carboxymethylcellulose, prolamine sulfate, or lactide/glycolide copolymers. The rate of release of the glutamine component may be controlled by altering the concentration of the macromolecule.

Sterile injectable solutions can be prepared by incorporating the glutamine compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the glutamine compound may be applied in pure form. However, it will generally be desirable to administer the glutamine compound to the skin as a composition or a formulation, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the glutamine compound to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508). Useful dosages of the glutamine composition can be determined by comparing their in vitro activity, and in vivo activity in animal models. Methods for the extrapolation of effective dosages in mice, and other animals, to humans are known to the art; for example, see U.S. Pat. No. 4,938,949.

For internal infections, the compositions can be administered orally or parenterally at dose levels of about 0.1 to 300 mg/kg, preferably 1.0 to 30 mg/kg of mammal body weight, and can be used in humans in a unit dosage form, administered one to four times daily in the amount of 1 to 1000 mg per unit dose.

For parenteral administration or for administration as drops, as for eye infections, the compounds are presented in aqueous solution in a concentration of from about 0.1 to about 10%, more preferably about 0.1 to about 7%. The solution may contain other ingredients, such as emulsifiers, antioxidants, or buffers.

Generally, the concentration of the glutamine compound in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The exact regimen for administration of the glutamine compositions disclosed herein will necessarily be dependent upon the needs of the individual subject being treated, the type of treatment and, of course, the judgment of the attending practitioner.

The antiviral activity of a glutamine compound of the invention can be determined using pharmacological models which are well known to the art.

The glutamine compositions are useful as anti-herpesvirus agents. Thus, they are useful to combat herpesvirus infections in animals, including man, and reactivation of herpesvirus following infection. The glutamine compounds are generally active against herpesviruses, and are particularly useful against the varicella zoster virus, the Epstein-Barr virus, the herpes simplex virus, the human herpesvirus type 8 (HHV-8) and the cytomegalovirus (CMV).

The glutamine composition of the present invention may be administered as a single agent therapy or in addition to an established therapy, such as inoculation with live, attenuated, or killed virus, or any other therapy known in the art to treat herpesvirus infection or reactivation.

Immunotherapeutic Applications

In further aspects of the present invention, the compositions described herein may be used for immunotherapy of herpesvirus infections. Within such methods, pharmaceutical compositions and vaccines are typically administered to a subject in need thereof, e.g., a human patient. The above pharmaceutical compositions and vaccines may be used to prophylactically prevent or ameliorate the extent of infection or reactivation by herpesvirus. Administration may be by any suitable method, including administration by intravenous, intraperitoneal, intramuscular, subcutaneous, intranasal, intradermal, anal, vaginal, topical, and oral routes.

Within certain embodiments, immunotherapy may be active immunotherapy, in which treatment relies on the in vivo stimulation of the endogenous host immune system to react against herpesvirus infection with the administration of immune response-modifying agents.

Within other embodiments, immunotherapy may be passive immunotherapy, in which treatment with glutamine also involves the delivery of agents with established herpesvirus-immune reactivity (such as effector cells or antibodies) that can directly or indirectly mediate therapeutic effects and does not necessarily depend on an intact host immune system. Cells for use in the compositions of the invention may generally be prepared in vitro or ex vivo, using standard procedures. Examples of effector cells include T lymphocytes (such as CD8+ cytotoxic T lymphocytes and CD4+ T-helper lymphocytes), killer cells (such as Natural Killer cells and lymphokine-activated killer cells), B cells, and antigen-presenting cells (such as dendritic cells and macrophages).

T cells for use in compositions of the invention may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures. T cells may be stimulated with a herpesvirus polypeptide, a polynucleotide encoding a herpesvirus polypeptide, or an antigen presenting cell (APC) that expresses such a polypeptide. Such stimulation is performed under conditions and for a time sufficient to permit the generation of T cells that are specific for the polypeptide. In certain embodiments, the herpesvirus polypeptide or polynucleotide is present within a delivery vehicle, such as a microsphere, to facilitate the generation of specific T cells that can be used in compositions of the present invention.

T cells are considered to be specific for a herpesvirus polypeptide if the T cells specifically proliferate, secrete cytokines, or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a herpesvirus polypeptide (100 ng/ml-100 μg/ml, preferably 200 ng/ml-25 μg/ml) for 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a herpesvirus polypeptide, polynucleotide, or polypeptide-expressing APC may be CD4+ or CD8+ herpesvirus protein-specific T cells. Within preferred embodiments, the T cells are derived from a patient, a related donor, or an unrelated donor, and are administered to the patient following stimulation and expansion.

Effector cells may generally be obtained in sufficient quantities for adoptive immunotherapy by growth in vitro. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Routes and frequency of administration of the therapeutic compositions described herein, as well as dosage, will vary from individual to individual, but may be readily established using standard techniques. In one embodiment, between 1 and about 10 doses may be administered over a 52 week period. In another embodiment, about 6 doses are administered, at intervals of about 1 month, and booster vaccinations are typically be given periodically thereafter. Alternate protocols may be appropriate for individual patients. In general, an appropriate dosage and treatment regimen provides the glutamine and other active compound(s) or cells in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a herpesvirus protein may correlate with an improved clinical outcome. Such immune responses may generally be evaluated using standard proliferation, cytotoxicity, or cytokine assays, which may be performed using samples obtained from a subject before and after treatment.

Dosages

The usual dose of orally administered glutamine for the various applications mentioned above is 0.5-0.57 grams/kilogram of body weight, which is about 25-30 grams per day for an adult who has low muscle mass (e.g., body weight of only 50 kg, about 110 pounds). Recommended adult doses of glutamine taken orally range from as little as 5 grams per day (roughly matching the dietary levels) to about 120 grams per day. The dosing is partly determined by body weight, with doses of 0.1-0.8 grams/kg being given according to various recommendations. Because glutamine is efficiently absorbed in the small intestine, blood levels reach a peak within an hour after ingestion.

Glutamine, which is available as a bulk powder that is essentially tasteless, can be easily dissolved in water, juice, or a blender drink and administered to the patient. Glutamine is metabolized to other amino acids, including glutamate, alanine (the second most abundant amino acid in skeletal muscle), citrulline, and arginine; in leukocytes, it can ultimately be metabolized to carbon dioxide. Following administration of glutamine, the increased blood content of the various amino acids that arise from glutamine metabolism return to baseline after a few hours (about four hours with high dose administration). It is possible that some liver or kidney diseases may lead to difficulties in metabolism of glutamine, so that administration of the substance in high doses should only be undertaken after adequate evaluation of the patient's condition and with careful monitoring of the responses to the glutamine administration. When administered at high doses over a long period of time (e.g., for several consecutive days), the body's own production of glutamine declines in compensation. As a result, the blood levels of glutamine, though higher than they were prior to supplementation, do not rise beyond a certain point because of the compensation by lower production rates.

The provision of sufficient intracellular electrolytes, principally potassium, magnesium, and phosphate, is required for optimum utilization of administered glutamine. Approximately 60-180 mEq of potassium, 10-30 mEq of magnesium, and 1040 mmole of phosphate per day appear necessary to achieve optimum metabolic response. In addition, sufficient quantities of the major extracellular electrolytes sodium, calcium, and chloride, must be given. Sodium and potassium may be added as the acetate salts to provide bicarbonate precursor.

The preferred dosage to be administered is likely to depend upon the type and extent of progression of the herpes infection being addressed, the overall health of the patient, and the route of administration. For topical and oral administration, formulations and dosages can be similar to those used for other anti-herpes drugs, e.g., acyclovir.

Antiviral Activity Assays

Glutamine, or derivatives, conjugates, and analogs thereof, for use in the invention can be tested for their effectiveness against infection by or reactivation of a member of the herpesvirus family. Anti-herpes simplex virus 1 (HSV-1) activity can be determined using a yield reduction assay utilizing a recombinant HSV (HSV US3:: pgC-lacZ) which expresses E. coli β-galactosidase (β-gal) under the control of an HSV late gene promoter (Fink, D. J.; Sternberg, L. R.; Weber, P. C.; Mata, M; Goins, W. F.; Glorioso, J. C. Human Gene Therapy 3:11-19, 1992). Vero (African Green Monkey kidney) cells are infected at a multiplicity of infection of 0.01 with the virus, and serial dilutions of the compound in dimethyl sulfoxide (DMSO) are added. The final concentration of DMSO in all wells is 1%. DMSO is added to control wells. The infection is allowed to proceed for 2 days at which time the β-gal activity in cell lysates is measured. Activity in wells containing compound is compared to control wells and percent inhibition determined. The EC₅₀ is defined as the concentration of drug that produces a 50% reduction in β-gal production relative to control wells.

Anti-human cytomegalovirus (HCMV) activity is determined using a yield reduction assay utilizing a recombinant HCMV (RC256) that produces β-gal (Spaete, R. R.; Mocarsid, E. S. Proceedings of the National Academy of Sciences USA 84:7213-7217, 1987). Primary human diploid fibroblasts (HFF cells) are infected at a moi of 0.01 with RC256, and serial dilutions of the compound in DMSO are added. The final concentration of DMSO in all wells is 1%. The infection is allowed to proceed for 7 days at which time the β-gal activity in cell lysates is measured. Activity in wells containing compound is compared to control wells and percent inhibition determined. The EC₅₀ is defined as the concentration of compound that produces a 50% reduction in P-gal production relative to control wells. TC₅₀ is defined as concentration of compound that produces cytotoxicity in 50% of uninfected cells.

Secondary yield reduction assays can also be used to determine the activity of glutamine derivatives, conjugates, and analogs against HSV. Vero cells are plated in 6 well dishes at a density of 5×10⁵ cells/well. Cells are infected at a multiplicity of infection of 0.01 with HSV (strain Syn17+). 30 μL of one of six threefold serial dilutions of the glutamine derivative, conjugate, or analog to be tested in DMSO is added to each well at the time of infection. The plates are returned to a 37° C. incubator and the infection is allowed to proceed for 2 days. Aliquots of the supernatant are harvested, and the virus titer is determined. Vero cells in 24 well plates are infected with threefold serial dilutions of supernatant. The virus is allowed to absorb to the monolayer for 1.5 hours, after which it is aspirated and replaced with growth medium containing 0.5% methylcellulose. Plaques are allowed to develop for 5 days, at which time the medium aspirated and the monolayer stained with crystal violet. The plaques are enumerated under low power magnification. Percent inhibition is determined by comparison with the titer from cells infected in the presence of DMSO alone.

To determine the activity of a glutamine derivative, conjugate, or analog against CMV, HFF cells, plated in 24 well plates at 1×10⁵ cells/well, are infected with CMV (strain AD169) at a moi of 0.01. 10 μL of one of six threefold dilutions of the glutamine derivative, conjugate, or analog to be tested in DMSO is added to each well at the time of infection. The plates are returned to a 37° C. incubator and the infection allowed to proceed for 7 days. Aliquots of the supernatant of infected cells are harvested and the virus titer determined. HFF cells in 24 well plates are infected with threefold serial dilutions of supernatant. The virus is allowed to adsorb to the cells for 2 hours, at which time the inoculum is aspirated and replaced with growth medium containing 0.5% methylcellulose. The plaques are allowed to develop for 7-10 days, at which time the medium is aspirated and the monolayer stained with crystal violet. The plaques are enumerated under low power magnification. Percent inhibition is determined by comparison with the titer from cells infected in the presence of DMSO alone.

Cellular toxicity assays: Cellular toxicity is measured in HFF cells. Cells are plated in 96 well plates at 1×10⁴ cells/well. Serial dilutions of a glutamine derivative, conjugate, or analog to be tested are added to the wells in DMSO, with the final concentration of DMSO in all wells at 1%, in a total volume of 200 μL. The plates are maintained in a 37° C. incubator for 7 days. 50 μL of a solution of XTT (sodium-3′[1-(phenyl-amino-carbonyl)-3,-tetrazolium]-bis(bis(4-methoxy-6-nitro)-benzene sulfonic acid hydrate) (3×10⁻⁴ mg/ml) is added to each well, and the plates returned to the incubator for 4 hours, after which the A₄₅₀ (absorbance at wavelength of 450 nm) for each well is measured in a plate reader. (Roehm, N. W., et al J. Immunol. Meth. 142:257-265, 1991). Toxicity is determined by comparison of the OD (optical density) of a well containing compound to the OD of wells containing DMSO only.

The effect of a glutamine derivative, conjugate, or analog to be tested on cellular DNA synthesis is measured in a ¹⁴C-thymidine incorporation assay, utilizing scintillation proximity assay technology. Cells are plated at 2×10⁴ cells/well in Amersham Cytostar 96 well scintillating microplates. The following day, serial dilutions of test compounds in DMSO are added to the wells, along with 0.1 μCi/well of [Methyl-¹⁴C]-thymidine (specific activity 50-62 mCi/mmol). The plates are counted immediately in a μBeta scintillation counter (Wallac), to determine background, then placed in a 37° C. incubator for 7 days. The plates are removed from the incubator at intervals and the thymidine incorporation into the cellular DNA determined by scintillation counting. Percent inhibition is determined by comparing ¹⁴C incorporation in wells containing the glutamine derivative, conjugate, or analog to be tested to incorporation in wells containing DMSO only.

Detecting Herpesvirus Infection

Methods of assaying for the presence of a herpesvirus infection, in particular HSV-1 or HSV-2 infection, in a subject, e.g., a vertebrate animal, such as a human, are known in the art. One method involves: (a) obtaining an antibody directed against a herpesvirus antigen; (b) obtaining a sample from a subject; (c) admixing the antibody with the sample; and (d) assaying the sample for antigen-antibody binding, wherein the antigen-antibody binding indicates herpesvirus infection in the subject. The assaying of the sample for antigen-antibody binding may be by precipitation reaction, radioimmunoassay, ELISA, Western blot, immunofluorescence, or any other method known to those of skill in the art.

Another method of assaying for the presence of a herpesvirus infection comprises: (a) obtaining a peptide containing an epitope that is known to be exposed on the surface of the virus or a cell infected with the virus, and which elicts an immune response by the subject infected with the virus; (b) obtaining a sample from the subject, such as a blood or serum sample; (c) admixing the peptide with the sample; and (d) assaying the sample for antigen-antibody binding, wherein the antigen-antibody binding indicates exposure of the subject to a herpesvirus. The assaying of the sample for antigen-antibody binding may be by precipitation reaction, radioimmunoassay, ELISA, Western blot, immunofluorescence, or any other method known to those of skill in the art.

Yet another method of assaying for the presence of a herpesvirus infection in an subject comprises: (a) obtaining an oligonucleotide probe comprising a sequence known to be associated with the herpesvirus genome; and (b) employing the probe in a PCR or other detection protocol known to those skilled in the art.

Herpesvirus infection may also, or alternatively, be detected based on the presence of T cells that specifically react with a herpesvirus protein in a biological sample. Within certain methods, a biological sample comprising CD4+ and/or CD8+ T cells isolated from a subject is incubated with a herpesvirus polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a subject by routine techniques (such as by Ficoll/Hypaque density gradient centrifugation of peripheral blood lymphocytes). T cells may be incubated in vitro for about 2-9 days (typically about 4 days) at 37° C. with polypeptide (e.g., 5-25 μg/ml). It may be desirable to incubate another aliquot of a T cell sample in the absence of herpesvirus polypeptide to serve as a control. For CD4+ T cells, activation is preferably detected by evaluating proliferation of the T cells. For CD8+ T cells, activation is preferably detected by evaluating cytolytic activity. A level of proliferation that is at least two fold greater and/or a level of cytolytic activity that is at least 20% greater than in disease-free subjects indicates the presence of herpesvirus infection in the patient.

As noted above, herpesvirus infection may also, or alternatively, be detected based on the level of mRNA encoding a herpesvirus protein in a biological sample. For example, at least two oligonucleotide primers may be employed in a polymerase chain reaction (PCR) based assay to amplify a portion of a herpesvirus cDNA derived from a biological sample, wherein at least one of the oligonucleotide primers is specific for (i.e., hybridizes to) a polynucleotide encoding the herpesvirus protein. The amplified cDNA is then separated and detected using techniques well known in the art, such as gel electrophoresis. Similarly, oligonucleotide probes that specifically hybridize to a polynucleotide encoding a herpesvirus protein may be used in a hybridization assay to detect the presence of polynucleotide encoding the herpesvirus protein in a biological sample.

To permit hybridization under assay conditions, oligonucleotide primers and probes should comprise an oligonucleotide sequence that has at least about 60%, preferably at least about 75% and more preferably at least about 90% or 95% or more, identity to a portion of a polynucleotide encoding a herpesvirus protein that is at least 10 nucleotides, and preferably at least 20, 30, or 40 nucleotides, in length. Preferably, oligonucleotide primers and/or probes hybridize to a polynucleotide encoding a polypeptide described herein under moderately stringent conditions, or under hybridization conditions known in the art. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test subject and from an individual who is not infected with herpesvirus. The amplification reaction may be performed on several dilutions of cDNA, for example spanning two orders of magnitude.

To improve sensitivity, multiple herpesvirus protein markers may be assayed within a given sample. It will be apparent that binding agents specific for different herpesvirus polypeptides may be combined within a single assay. Further, multiple primers or probes may be used concurrently. The selection of herpesvirus protein markers may be based on routine experiments to determine combinations that results in optimal sensitivity. In addition, or alternatively, assays for herpesvirus proteins provided herein may be combined with assays for other known herpesvirus antigens.

The invention is further described in the following non-limiting examples.

### EXAMPLES

### Example 1

### Materials and Methods

Virus and Virological Analyses

The KOS strain of HSV-1 is propagated in Vero cells (ATCC No. CCL-81, American Type Culture Collection, Manassass, Va.), titered by viral plaque assay, and stored at −70° C. until used to infect animals.

Ocular swabs are immersed in 0.2 ml of complete tissue culture medium consisting of RPMI-1640, 10% fetal bovine serum (FBS), and an antibiotic-antimycotic mixture (Life Technologies, Grand Island, N.Y.). The swabs in culture medium are stored at 4° C. until testing by plaque assay. The determination of infectious virus in the cornea and in the trigeminal ganglia of animals is performed in a similar manner. Pairs of corneas and ganglia from individual mice are placed in separate tubes in 0.2 ml of complete medium, homogenized, clarified by centrifugation at 14,000×g for 5 minutes and the supernatant tested for infectious virus on Vero cells.

Animals and Animal Infection. Male BALB/c strain mice (The Jackson Laboratory, Bar Harbor, Me.) are infected at 5 weeks of age by topical application of 5×10⁵ plaque forming units of the KOS strain of HSV-1 following superficial scratching of the cornea. Documentation of the success of viral infection is determined by culturing ocular swabs taken on days 3 and 5 after infection. Animals without infectious virus on both days 3 and 5 in both eyes are excluded from use. Thirty days after infection the eyes are swabbed to establish that infectious virus is no longer present on the ocular surface. Groups of uninfected age and sex-matched mice are used as control animals.

Quantitation of Viral DNA. Quantitation of herpes viral DNA in tissue homogenates is made using the polymerase chain reaction (PCR). In brief, the procedure involves comparing DNA extracted from experimental and control tissue samples containing herpes viral DNA, and performing serial dilutions with DNA from homologous tissue homogenates of uninfected animals. Using a log dilution series and previously determined quantities of herpes viral DNA, it is possible to obtain a series of curves which permit the sensitive and reproducible determination of herpes viral DNA in experimental samples. The sensitivity of this procedure permits the determination of differences of 100 herpes viral genome equivalents per sample. In each experimental series, standard curves are conducted along with the analyses of the tissue homogenates from experimental (drug treated) and control (placebo treated) mice.

### Example 2

### Effect of Glutamine on Heat-Stress Reactivation of HSV

To test the effect of glutamine (or a composition containing a derivative, conjugate, or analog of glutamine) on reactivation of HSV induced by heat stress, two sets of experiments can be performed. In one set, groups of mice with latent HSV are immersed in 43° C. water up to their necks for 10 minutes. Following the hyperthermic stress, the animals are dried and given an intraperitoneal (IP) injection of 0.1 mg of glutamine suspended in 0.1 ml saline. At 8 and 16 hours after hyperthermia, the animals are again treated with glutamine. Control mice are given IP injections of saline on the same schedule. At 24 hr after hyperthermic stress, the eyes are swabbed, the mice are sacrificed, and the corneas and trigeminal ganglia are analyzed for infectious virus and viral DNA.

Mice can also be treated by IP injection for 3 days prior to hyperthermic stress at 8 hr intervals on each of the 3 days. The animals are then heat stressed, treated immediately after stress with glutamine, and treated at 8 and 16 hr after hyperthermic stress. Control mice are given IP injections of saline on the same schedule. Two additional groups of mice are prophylactically treated by oral administration of 0.1 mg glutamine or saline on the same dose schedule. In each experiment, animals are subdivided into those which are hyperthermically stressed and those which are not stressed. As above, the eyes of these animals are swabbed at 24 hr after stress and then sacrificed for analysis of infectious virus and viral DNA in their corneas and trigeminal ganglia

Control mice include animals which are latent for HSV-1 but which are not heat stressed. Subsets of this control group may include animals placebo-treated and animals treated with glutamine. Two additional control groups of animals are those which are not latent for HSV-1, but which are either treated with placebo or glutamine on the schedules outlined above.

The body temperature of glutamine-treated and saline-treated mice can be determined before and after heat stress with a microprobe thermometer and rectal probe (World Precision Instruments, Sarasota, Fla.) to confirm that the administration of glutamine does not produce an effect on body temperature.

### Example 3

### Effect of Glutamine Prophylaxis on Viral Reactivation

To test for a prophylactic effect of glutamine (or a composition containing a derivative, conjugate, or analog of glutamine), mice can be treated either IP, orally, or topically with glutamine for three days prior to receiving the reactivation hyperthermic stimulus. These mice can be tested for the levels of infectious virus in their ocular tear film compared to placebo-treated mice which undergo hyperthermically induced reactivation. Control animals that are latent for virus but not heat-stressed and animals that are heat-stressed but not infected should not have infectious virus in their ocular tear film.

Homogenates of the corneas and trigeminal ganglia of mice that are prophylactically treated with glutamine can also be tested for the level of infectious units as compared to homogenates from placebo-treated animals that are heat-stressed. Again, infectious virus should not be present in the homogenates of control animals that are latent for virus but not heat-stressed and animals that are heat-stressed but not infected.

### Example 4

### Viral DNA Concentration in the Cornea and Trigeminal Ganglion

The levels of viral DNA found in the corneas of animals treated both before and after heat stress with glutamine (or a composition containing a derivative, conjugate, or analog of glutamine) can be tested to determine whether these levels are different from the levels found in either placebo-treated animals or latent animals which are not heat-stressed. Viral DNA should not be found in control mice that are not latently infected regardless of whether or not they were heat-stressed.

### Example 5

### Effect of Glutamine on Viral Reactivation

To test the effect of glutamine (or a composition containing a derivative, conjugate, or analog of glutamine) on reactivation of HSV, female BALB/c mice (The Jackson Laboratory, Bar Harbor, Me.) at 4 weeks of age can be used. The strain of HSV and the method of propagation can be as described in Example 1.

The mice can be infected by, e.g., the topical ocular route, and the establishment of a corneal infection can be documented by slit lamp examination and ocular swabs on day 3 after infection. Only animals which are successfully infected are retained for use in these experiments. Thirty days after infection, groups of latently infected and uninfected mice are treated with the glutamine composition. The dose of glutamine sufficient to prevent or reduce viral reactivation can be determined in a preliminary experiment in which groups of mice are injected intraperitoneally with varying concentrations of glutamine, e.g., 0.1, 0.5, 1.0, and 5.0 mg of glutamine four times daily. The mice are treated one day before heat stress and the day of the heat stress. The most effective dose in preventing viral reactivation can then be determined.

Two days prior to hyperthermic stress, uninfected mice and latently infected mice can be treated four times daily with glutamine at a concentration found to be effective using the methods described above. The glutamine composition can be given intraperitoneally in some groups of mice and orally in others. This treatment can be continued on the day the animals are heat stressed by immersion into 43° C. water for 10 minutes. Twenty-four hours after the hyperthermic stress, the eyes are swabbed to culture any infectious virus, and the mice killed to collect the trigeminal ganglia. Ocular swabs are incubated in culture medium which is then transferred to Vero cell monolayers in 96 well culture plates. Units of infectious herpesvirus are recorded as units of cytopathic effect over a period of seven days. Units of cytopathic effect are the number of plaques or dead Vero cells caused by the virus, i.e., one viral particle causes one unit of cytopathic effect (a plaque.). Pairs of trigeminal ganglia from each mouse are homogenized in 0.5 ml of tissue culture medium, and the homogenate can be tested for infectious virus by adding to Vero cell monolayers. The units of cytopathic effect are recorded over a seven day incubation period.

Because the glutamine can be administered by two different routes, intraperitoneally and orally, differences between these treatment approaches should be included in the statistical analysis. The mean number of animals exhibiting reactivation in each of the groups can be compared by a one way analysis of variance.

Treatment of animals with glutamine intraperitoneally or orally can be shown to prevent or reduce the frequency with which infectious herpesvirus is present on the ocular surface. Compared to untreated animals that are heat-stressed, glutamine treatment by either route results in statistically significantly lower units of infectious herpesvirus. Control, uninfected mice that are heat-stressed do not indicate any virus on their ocular surfaces.

### Example 6

### Inhibition of HSV Reactivation Using a Squirrel Monkey Model

The corneas of squirrel monkeys are infected with HSV-1 (strain Rodanus) as described by Varnell et al., Invest. Opthalmol. Vis. Sci., 36:1181-1183 (1995). All corneas show typical herpetic dendritic lesions 3 or 4 days after infection; 15 days after infection, all corneas are typically clear. The monkeys are divided into two groups. The first group receives a composition containing glutamine (or a composition containing a derivative, conjugate, or analog of glutamine), while the second group receives vehicle alone, both by the intraperitoneal route. Treatment is administered over 25 days to both groups. The animals are neither tranquilized nor anesthetized for the treatments, but only hand restrained. All corneas are stained with sodium fluorescein and examined daily each weekday over the 25-day treatment period (total examination days=18) and for 25 days after cessation of treatment (total additional examination days=18) by an observer masked as to the treatment group.

Observed lesions are typically true recurrences in that the earlier lesions will have completely healed and the new lesions develop in slightly different areas. Results of these experiments are reported as the ratio of the number of eyes with recurrent disease to the total number of eyes, and statistical significance is determined by one-tailed chi-squared analysis. In addition, cumulative total recurrences in both the treatment and control groups is compared, both during and following cessation of treatment.

### Example 7

### Administration of Glutamine to a Patient to Prevent Herpesvirus Reactivation

A human patient diagnosed as being infected with a herpesvirus can be administered a glutamine-containing composition (e.g., a composition containing glutamine, or a derivative, conjugate, or analog of glutamine) according to the invention for the prevention or treatment of reactivation of a herpesvirus infection caused by factors, such as stress, and the associated complications and outcomes. The patient can prophylactically apply the glutamine-containing composition topically to an area prone to develop lesions or blisters, such as the eyes, the genitals, and the lips and mouth, or the patient can topically apply the glutamine composition to an area in which the virus is active or has already reactivated and in which lesions or blisters have already developed. The patient may also choose to orally administer the glutamine-containing composition. Administration of the glutamine-containing composition will act to prevent the development of lesions or blisters associated with herpesvirus reactivation when applied prophylactically, and will increase the healing and resolution of diseases or conditions associated with herpesvirus reactivation.

In cases where the disease or condition to be treated or prevented includes, e.g., encephalitis, shingles, and cancer (e.g., lymphomas, such as Burkitt's lymphoma, nasopharyngeal carcinoma, Hodgkins disease, Karposi's sarcoma, and multiple myeloma), which is caused by reactivation of a herpesvirus, the patient can be administered the glutamine-containing composition parenterally, such as by intraperitoneal, intravenous, intraarterial, intramuscular, intracranial, subcutaneous, intradermal, or ocular injection, or by inhalation.

### OTHER EMBODIMENTS

The complete disclosures of all references cited in this specification, including U.S. Provisional Application No. 60/703,835, are hereby incorporated by reference. While the forms of the invention herein disclosed constitute presently preferred embodiments, many others are possible. It is not intended herein to mention all of the possible equivalent forms or ramifications of the invention. It is understood that the terms used herein are merely descriptive, rather than limiting, and that various changes may be made without departing from the spirit or scope of the invention.

What is claimed is:

1-65. (canceled)

66. A composition comprising an amount of glutamine or a derivative, conjugate, or analog thereof that is sufficient for preventing or reducing reactivation of a latent herpesvirus infection in combination with a pharmaceutically acceptable carrier.

67. The composition of claim 1, which is packaged for parenteral, oral, or topical use by a patient.

68. The composition of claim 1, wherein said packaging further comprises instructions for the administration of said composition for treating or preventing reactivation of a herpesvirus infection.

69. The composition of claim 1, wherein said composition is formulated as a cream, lotion, gel, ointment, plaster, stick, pen, injection, or tablet.

70. The composition of claim 1, wherein said pharmaceutically acceptable carrier is selected from sterile water, saline, polyalkylene glycols, vegetable oils, hydrogenated naphthalenes, biocompatible polymers, biodegradable polymers, and mixtures thereof.

71. The composition of claim 1, wherein said composition further comprises arginine, methionine, or derivatives, conjugates, or analogs thereof.

72. The composition of claim 1, wherein said composition further comprises an inhibitor of herpesvirus thymidine kinase, lysine, or an antiherpesvirus substance selected from a pre-phosphorylated or phosphonate nucleoside analog, a pyrophosphate analog, and a nucleoside analog, or any combination thereof, or an ester, salt, or solvate thereof.

73. The composition of claim 72, wherein the thymidine kinase inhibitor is selected from azidodeoxythymidine, didehydrodeoxythymidine, 2-phenylamino-9-substituted-6-oxopurines, and 2-phenylamino-9H-6-oxopurines, or a ester, salt, or solvate thereof.

74. The composition of claim 72, wherein said pre-phosphorylated or phosphonate nucleoside analog is selected from acyclovir monophosphate, ganciclovir monophosphate, and 9-(phosphonomethoxyethyl)adenine (PMEA), or an ester, salt, or solvate thereof.

75. The composition of claim 72, wherein said pyrophosphate analog is selected from phosphonoacetate and phosphonoformate.

76. The composition of claim 72, wherein said nucleoside analog is selected from acyclovir, ganciclovir, cidofovir, and famcyclovir.

77. A method of treating or preventing the reactivation of a latent herpesvirus infection in a subject, said method comprising administering to said subject a therapeutically effective amount of the composition of claim 66.

78. The method of claim 77, wherein the latent herpesvirus infection is an infection caused by a herpesvirus selected from a group consisting of herpes simplex virus (HSV) type 1 (HSV-1), HSV-2, cytomegalovirus (CMV), Epstein Barr virus (EBV), varicella zoster virus (VZV), human herpes virus (HHV)-6, HHV-7, and HHV-8.

79. The method of claim 77, wherein the latent herpesvirus infection is an infection of the eyes, mouth, lips, genital area, or anal area of the subject.

80. The method of claim 77, wherein said infection is in the skin, a mucous membrane, or the neurological system.

81. The method of claim 77, wherein the subject is a human.

82. The method of claim 77, wherein said administering is performed by injection, oral administration, or topical administration.

83. The method of claim 77, wherein said composition is administered topically to the eyes, mouth, lips, genital area, anal area, or combinations thereof of said subject.

84. The method of claim 77, further comprising diagnosing said subject with a herpesvirus infection prior to said administering.

85. A composition comprising an amount of arginine or methionine, or derivatives, conjugates, or analogs thereof, that is sufficient for preventing or reducing reactivation of a latent herpesvirus infection in combination with a pharmaceutically acceptable carrier.

86. The composition of claim 85, wherein said composition further comprises glutamine, or derivatives, conjugates, or analogs thereof.

87. The composition of claim 85, which is packaged for parenteral, oral, or topical use by a patient.

88. The composition of claim 87, wherein said packaging further comprises instructions for the administration of said composition for treating or preventing reactivation of a herpesvirus infection.

89. The composition of claim 85, wherein said composition further comprises an inhibitor of herpesvirus thymidine kinase, lysine, or an antiherpesvirus substance selected from a pre-phosphorylated or phosphonate nucleoside analog, a pyrophosphate analog, and a nucleoside analog, or any combination thereof, or an ester, salt, or solvate thereof.

90. The composition of claim 89, wherein the thymidine kinase inhibitor is selected from azidodeoxythymidine, didehydrodeoxythymidine, 2-phenylamino-9-substituted-6-oxopurines, and 2-phenylamino-9H-6-oxopurines, or a ester, salt, or solvate thereof.

91. The composition of claim 89, wherein said pre-phosphorylated or phosphonate nucleoside analog is selected from acyclovir monophosphate, ganciclovir monophosphate, and 9-(phosphonomethoxyethyl)adenine (PMEA), or an ester, salt, or solvate thereof.

92. The composition of claim 89, wherein said pyrophosphate analog is selected from phosphonoacetate and phosphonoformate.

93. The method of claim 89, wherein said nucleoside analog is selected from acyclovir, ganciclovir, cidofovir, and famcyclovir.

94. A method of treating or preventing the reactivation of a latent herpesvirus infection in a subject, said method comprising administering to said subject a therapeutically effective amount of the composition of claim 85.

95. The method of claim 94, wherein the latent herpesvirus infection is an infection caused by a herpesvirus selected from a group consisting of herpes simplex virus (HSV) type 1 (HSV-1), HSV-2, cytomegalovirus (CMV), Epstein Barr virus (EBV), varicella zoster virus (VZV), human herpes virus (HHV)-6, HHV-7, and HHV-8.

96. The method of claim 94, wherein the latent herpesvirus infection is an infection of the eyes, mouth, lips, genital area, or anal area of the subject.

97. The method of claim 94, wherein said infection is in the skin, a mucous membrane, or in the neurological system.

98. The method of claim 94, wherein the subject is a human.

99. The method of claim 94, wherein said administering is performed by injection, oral administration, or topical administration.

100. The method of claim 94, wherein said composition is administered topically to the eyes, mouth, lips, genital area, anal area, or combinations-thereof of said subject.

101. The method of claim 94, further comprising diagnosing said subject with a herpesvirus infection prior to said administering.
